(19) United States
(12) Patent Application Publication
(10) Pub. No.: US 2007/0244111 A1
(43) Pub. Date: Oct. 18, 2007

(54) Pyrazolopyrimidines

(75) Inventors: Olaf Gebauer, Leverkusen (DE); Herbert Gayer, Monheim (DE); Ulrich Heinemann, Leichlingen (DE); Stefan Herrmann, Langenfeld (DE); Stefan Hillebrand, Neuss (DE); Hans-Ludwig Elbe, Wuppertal (DE); Ronald Ebbert, Nurnberg (DE); Ulrike Wachendorff-Neumann, Neuwied (DE); Peter Dahmen, Neuss (DE); Karl-Heinz Kuck, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: BAYER CROPSCIENCE AKTIENGESELLSCHAFT, Monhiem (DE)

(21) Appl. No.: 10/581,945

(22) PCT Filed: Dec. 9, 2004
(86) PCT No.: PCT/EP04/13989

§ 371 Date: May 14, 2007

(30) Foreign Application Priority Data
Dec. 10, 2003 (DE) 10357565.0

(51) Int. Cl. A01N 43/90; A01N 43/54; C07D 231/54; C07D 413/14; C07D 487/04; C07D 239/70; A01N 43/84

(52) U.S. Cl. 514/233.2; 514/259.3; 544/117; 544/281

(57) ABSTRACT

Novel pyrazolopyrimidines of the formula 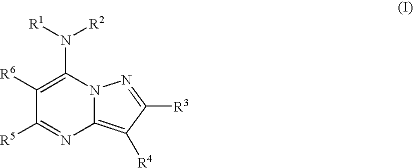 in which
R¹, R², R³, R⁴, R⁵ and R⁶ are as defined in the description, a plurality of processes for preparing these compounds and their use for controlling unwanted microorganisms.

The present invention relates to novel pyrazolopyrimidines, to a plurality of processes for their preparation and to their use for controlling unwanted microorganisms.

It is already known that certain pyrazolopyrimidines have fungicidal properties (compare DE-A 3 130 633 or FR-A 2 794 745).

However, since the ecological and economical demands made on modern fungicides are increasing constantly, for example with respect to activity spectrum, toxicity, selectivity, application rate, formation of residues and favorable manufacture, and there can furthermore be problems, for example, with resistance, there is a constant need to develop novel fungicides which, at least in some areas, have advantages over those of the prior art.

This invention now provides novel pyrazolopyrimidines of the formula 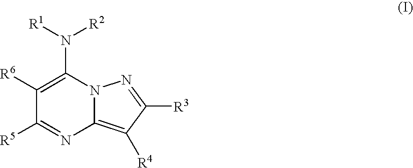
in which
R¹ represents optionally substituted alkyl, optionally substituted alkenyl, optionally substituted alkynyl, optionally substituted cycloalkyl or optionally substituted heterocyclyl,
R² represents hydrogen or alkyl, or
R¹ and R² together with the nitrogen atom to which they are attached represent an optionally substituted heterocyclic ring,
R³ represents hydrogen or alkyl,
R⁴ represents optionally substituted alkenyl or optionally substituted alkynyl,
R⁵ represents halogen, CN, alkyl, alkoxy or alkylthio and
R⁶ represents alkyl, cycloalkyl or optionally substituted aryl.

Depending on the substitution pattern, the compounds according to the invention can, if appropriate, be present as mixtures of different possible isomeric forms, in particular of stereoisomers, such as E and Z, threo and erythro and also optical isomers, and, if appropriate, also in the form of tautomers. If R⁶ is, at both atoms adjacent to the point of attachment, substituted by different substituents, the compounds in question may be present in a particular stereoisomeric form, i.e. as atropisomers.

Furthermore, it has been found that pyrazolopyrimidines of the formula (I) can be obtained when
a) pyrazolopyrimidines of the formula 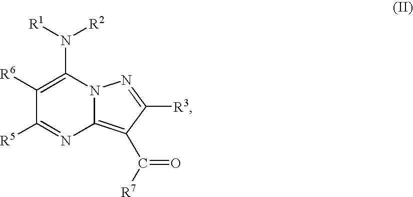
in which
   R¹, R², R³, R⁵ and R⁶ are as defined above and
   R⁷ represents hydrogen or alkyl are reacted with phosphonium salts of the formula
      Y₃⊕P—CH₂—R⁸X^{⊖} (III)
   in which
   Y represents alkyl, cycloalkyl, aralkyl or phenyl
   X represents an anion, such as bromide, and
   R⁸ represents hydrogen or optionally substituted alkyl in the presence of a base in the presence of a diluent, or
b) pyrazolopyrimidines of the formula 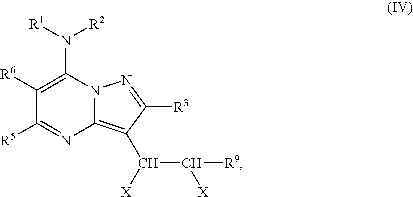
in which
   R¹, R², R³, R⁵ and R⁶ are as defined above,
   R⁹ represents hydrogen or optionally substituted alkyl,
   X represents chlorine or bromine are reacted with strong bases in the presence of a diluent, or
c) pyrazolopyrimidines of the formula 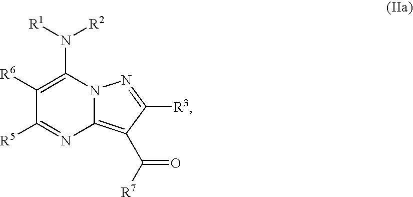
in which
   R¹, R², R³, R⁵, R⁶ and R⁷ are as defined above are initially reacted with phosphorus oxychloride in the presence of dimethylformamide and then further with a base to give a compound of the formula (V) 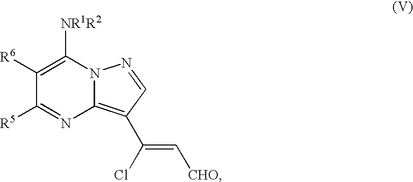
   or
d) pyrazolopyrimidines of the formula 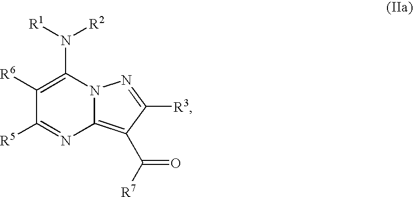
in which
   R¹, R², R³, R⁵, R⁶ and R⁷ are as defined above, are reacted with Grignard compounds
      R⁸—CH₂—Mg X,
   where R⁸ is as defined above, and then acidified.

Finally, it has been found that the pyrazolopyrimidines of the formula (I) are highly suitable for controlling unwanted microorganisms. In particular, they have strong fungicidal activity and can be used both in crop protection and in the protection of materials.

The formula (I) provides a general definition of the pyrazolopyrimidines according to the invention. Preference is given to those compounds of the formula (I) in which R⁴ represents optionally substituted alkenyl. Preference is also given to those compounds of the formula (I) in which R⁴ represents optionally substituted alkynyl. Preference is furthermore given to those compounds of the formula (I) in which
R¹ represents alkyl having 1 to 6 carbon atoms which may be mono- to pentasubstituted by identical or different substituents from the group consisting of halogen, cyano, hydroxyl, alkoxy having 1 to 4 carbon atoms and cycloalkyl having 3 to 6 carbon atoms, or
R¹ represents alkenyl having 2 to 6 carbon atoms which may be mono- to trisubstituted by identical or different substituents from the group consisting of halogen, cyano, hydroxyl, alkoxy having 1 to 4 carbon atoms and cycloalkyl having 3 to 6 carbon atoms, or
R¹ represents alkynyl having 3 to 6 carbon atoms which may be mono- to trisubstituted by identical or different substituents from the group consisting of halogen, cyano, alkoxy having 1 to 4 carbon atoms and cycloalkyl having 3 to 6 carbon atoms, or
R¹ represents cycloalkyl having 3 to 6 carbon atoms which may be mono- to trisubstituted by identical or different substituents from the group consisting of halogen and alkyl having 1 to 4 carbon atoms, or
R¹ represents saturated or unsaturated heterocyclyl having 5 or 6 ring members and 1 to 3 heteroatoms, such as nitrogen, oxygen and/or sulfur, where the heterocyclyl may be mono- or disubstituted by halogen, alkyl having 1 to 4 carbon atoms, cyano, nitro and/or cycloalkyl having 3 to 6 carbon atoms,
R² represents hydrogen or alkyl having 1 to 4 carbon atoms, or
R¹ and R² together with the nitrogen atom to which they are attached represent a saturated or unsaturated heterocyclic ring having 3 to 6 ring members, where the heterocycle may contain a further nitrogen, oxygen or sulfur atom as ring member and where the heterocycle may be substituted up to three times by fluorine, chlorine, bromine, alkyl having 1 to 4 carbon atoms and/or haloalkyl having 1 to 4 carbon atoms and 1 to 9 fluorine and/or chlorine atoms,
R³ represents hydrogen or alkyl having 1 to 4 carbon atoms,
R⁴ represents alkenyl having 2 to 6 carbon atoms or alkynyl having 2 to 6 carbon atoms, or
R⁴ represents alkenyl having 2 to 4 carbon atoms which is substituted by carboxyl, methoxycarbonyl or ethoxycarbonyl, or formyl or halogen, or
   represents alkynyl having 2 to 4 carbon atoms which is substituted by carboxyl, methoxycarbonyl or ethoxycarbonyl, formyl or halogen,
R⁵ represents fluorine, chlorine, bromine, CN, alkoxy having 1 to 4 carbon atoms or alkylthio having 1 to 4 carbon atoms and
R⁶ represents alkyl having 1 to 6 carbon atoms or represents cycloalkyl having 3 to 6 carbon atoms, or
   represents phenyl which may be mono- to tetrasubstituted by identical or different substituents from the group consisting of
   halogen, cyano, nitro, amino, hydroxyl, formyl, carboxyl, carbamoyl, thiocarbamoyl;
   in each case straight-chain or branched alkyl, alkoxy, alkylthio, alkylsulfinyl or alkylsulfonyl having in each case 1 to 6 carbon atoms;
   in each case straight-chain or branched alkenyl or alkenyloxy having in each case 2 to 6 carbon atoms;
   in each case straight-chain or branched haloalkyl, haloalkoxy, haloalkylthio, haloalkylsulfinyl or haloalkylsulfonyl having in each case 1 to 6 carbon atoms and 1 to 13 identical or different halogen atoms;
   in each case straight-chain or branched haloalkenyl or haloalkenyl oxy having in each case 2 to 6 carbon atoms and 1 to 11 identical or different halogen atoms;
   in each case straight-chain or branched alkylamino, dialkylamino, alkylcarbonyl, alkylcarbonyl oxy, alkoxycarbonyl, alkylsulfonyloxy, hydroximinoalkyl or alkoximinoalkyl having in each case 1 to 6 carbon atoms in the individual alkyl moieties;
   cycloalkyl having 3 to 6 carbon atoms,
   2,3-attached 1,3-propanediyl, 1,4-butanediyl, methylenedioxy (—O—CH₂—O—) or 1,2-ethylenedioxy (—O—CH₂—CH₂—O—), where these radicals may be mono- or polysubstituted by identical or different substituents from the group consisting of halogen, alkyl having 1 to 4 carbon atoms and haloalkyl having 1 to 4 carbon atoms and 1 to 9 identical or different halogen atoms.

Particular preference is given to those pyrazolopyrimidines of the formula (I) in which
R¹ represents a radical of the formula 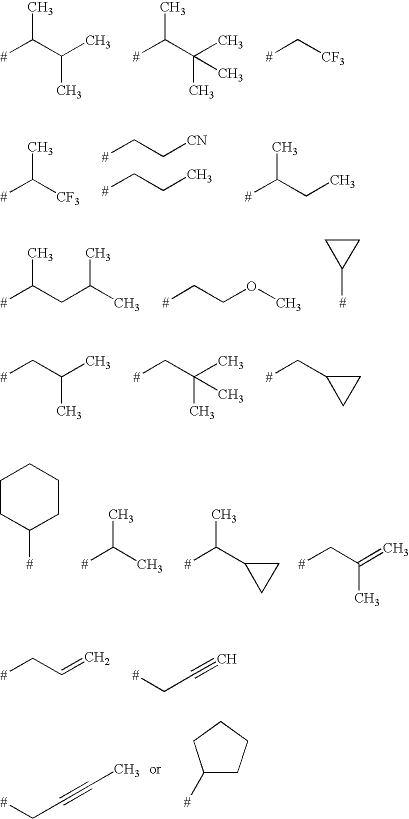
where # denotes the point of attachment and where for those radicals which may be present in optically active form each of the possible stereoisomers or mixtures thereof may be present,
R² represents hydrogen, methyl, ethyl or propyl, or
R¹ and R² together with the nitrogen atom to which they are attached represent pyrrolidinyl, piperidinyl, morpholinyl, thiomorpholinyl, piperazinyl, 3,6-dihydro-1(2H)-piperidinyl or tetrahydro-1(2H)-pyridazinyl, where these radicals may be substituted by 1 to 3 fluorine atoms, 1 to 3 methyl groups and/or trifluoromethyl, or
R¹ and R² together with the nitrogen atom to which they are attached represent a radical of the formula 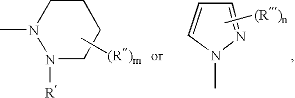
in which
R′ represents hydrogen or methyl,
R″ represents methyl, ethyl, fluorine, chlorine or trifluoromethyl,
m represents the number 0, 1, 2 or 3, where R″ represents identical or different radicals if m represents 2 or 3,
R′″ represents methyl, ethyl, fluorine, chlorine or trifluoromethyl and
n represents the number 0, 1, 2 or 3, where R′″ represents identical or different radicals if n represents 2 or 3,
R³ represents hydrogen, methyl, ethyl, propyl or isopropyl,
R⁴ represents straight-chain or branched alkenyl having 2 to 5 carbon atoms, where each of these radicals may be monosubstituted by carboxyl, methoxycarbonyl, ethoxycarbonyl, formyl or halogen, or
R⁴ represents alkynyl having 2 to 5 carbon atoms, where each of these radicals may be monosubstituted by carboxyl, methoxycarbonyl or ethoxycarbonyl,
R⁵ represents fluorine, chlorine, CN, methoxy, ethoxy, methylthio or ethylthio, and
R⁶ represents straight-chain or branched alkyl having 1 to 4 carbon atoms, represents cyclopropyl, cyclobutyl, cyclopentyl or cyclohexyl, or
R⁶ represents phenyl which may be mono- to trisubstituted by identical or different substituents from the group consisting of fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulfinyl, ethylsulfinyl, methylsulfonyl, ethylsulfonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulfinyl, trifluoromethylsulfonyl, trichloroethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinom ethyl, methoximinoethyl, ethoximinoethyl, cyclopropyl, cyclobutyl, cyclopentyl and cyclohexyl,
   2,3-attached 1,3-propanediyl, methylenedioxy (—O—CH₂—O—) or 1,2-ethylenedioxy (—O—CH₂—CH₂—O—), where these radicals may be mono- or polysubstituted by identical or different substituents from the group consisting of fluorine, chlorine, methyl, ethyl, n-propyl, i-propyl and trifluoromethyl.

A very particularly preferred group of compounds according to the invention are pyrazolopyrimidines of the formula (I) in which
R¹, R², R³ and R⁵ have the particularly preferred meanings given above,
R⁴ represents a radical of the formula 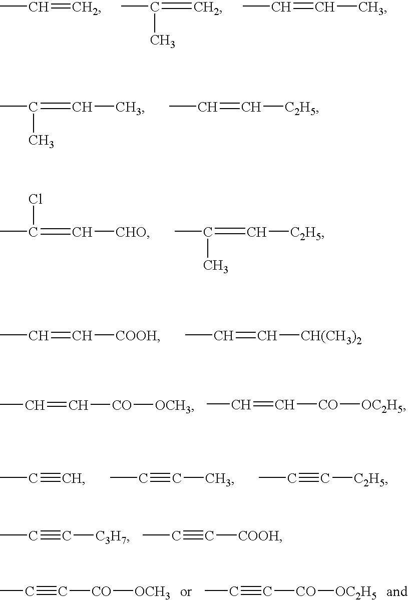
R⁶ represents methyl, ethyl, propyl, isopropyl, n-butyl, tert-butyl, cyclopropyl, cyclopentyl or cyclohexyl, or
R⁶ represents 2,4-, 2,5- or 2,6-disubstituted phenyl or 2-substituted phenyl or represents 2,4,6-trisubstituted phenyl, possible substituents being the radicals mentioned in the context of the enumeration of the particularly preferred definitions.

The radical definitions mentioned above can be combined with one another as desired. Moreover, individual definitions may not apply.

Using 3-formyl-5-chloro-6-(2-chloro-4-fluorophenyl)-7-(4-methylpiperidino)pyrazolo[1,5a]-pyrimidine as starting material and triphenylmethylphosphonium bromide as reaction component, the course of the process (a) according to the invention can be illustrated by the formula scheme below.

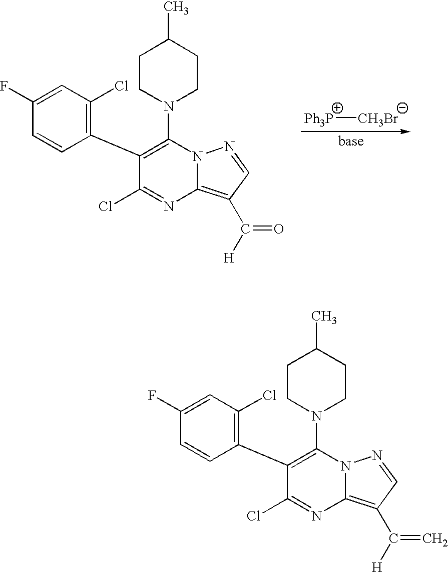

Using, for example, 3-formyl-5-chloro-6-(2-chloro-4-fluorophenyl)-7-(4-methylpiperidino)pyrazolo[1,5a]pyrimidine as starting material and bromomethyltriphenylphosphonium bromide as starting material, the corresponding alkyne is obtained:

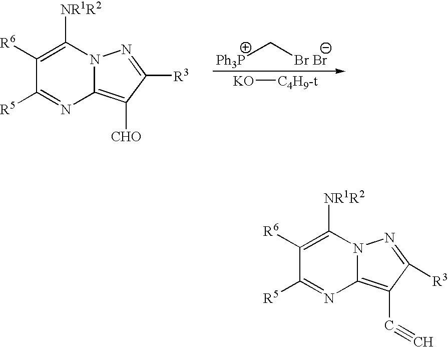

Using 3-(1,2-dibromopropyl)-5-chloro-6-(2-chloro-4-fluorophenyl)-7-(4-methylpiperidino)pyrazolo[1,5a]pyrimidine as starting material and potassium tert-butoxide as reaction component, the course of the process (b) according to the invention can be illustrated by the formula scheme below.

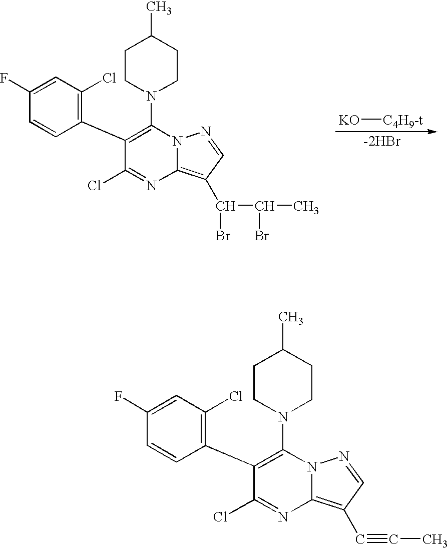

Using 3-acetyl-5-chloro-6-(2-chloro-4-fluorophenyl)-7-(4-methylpiperidino)pyrazolo[1,5a]pyrimidine as starting material, phosphorus oxychloride as reaction component in the first step and potassium tert-butoxide as base in the second step, the course of the process (c) according to the invention can be illustrated by the formula scheme below.

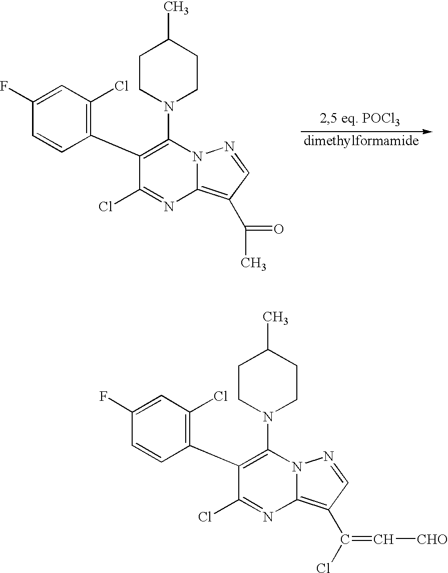

Using 3-formyl-5-chloro-6-(2-chloro-3-difluorophenyl)-7-(4-methylpiperidino)pyrazolo[1,5a]pyrimidine as starting material and methylmagnesium bromide as reaction component, the course of the process (d) according to the invention can be illustrated by the formula scheme below:

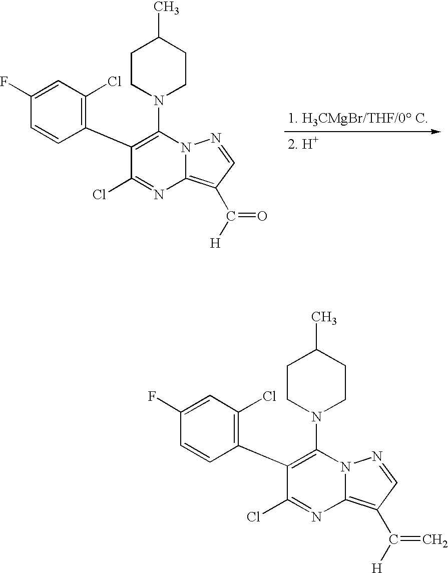

The formula (II) provides a general definition of the pyrazolopyrimidines required as starting materials for carrying out the process (a) according to the invention. In this formula, R¹, R², R³, R⁵ and R⁶ preferably have those meanings which have already been mentioned in connection with the description of the compounds of the formula (I) according to the invention as being preferred for these radicals. R⁷ preferably represents hydrogen, methyl or ethyl.

The pyrazolopyrimidines of the formula (II) are obtained when
e) cyano compounds of the formula 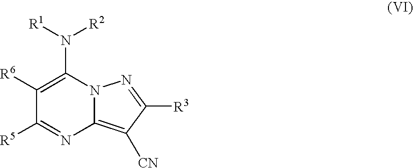
in which
   R¹, R², R³, R⁵ and R⁶ are as defined above are either
   α) reacted with diisobutylaluminum hydride in the presence of aqueous ammonium chloride solution and also in the presence of an organic diluent, or
   β) reacted with Grignard compounds of the formula
      R¹⁰—Mg—X¹ (VII)
   in which
      R¹⁰ represents alkyl and
      X¹ represents chlorine or bromine
      in the presence of a diluent and, if appropriate, in the presence of a catalyst,
f) pyrazolopyrimidines of the formula (VIII) 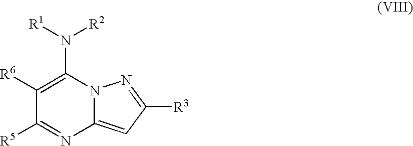
in which
   R¹, R², R³, R⁵ and R⁶ are as defined above are reacted with acid halides, acid anhydrides or other activated carboxylic acid derivatives of the formula (IX)
      R¹⁰—CO—X² (IX)
   in which
   R¹⁰ is as defined above and
   X² represents chlorine, bromine, a radical of the formula —O—CO—R¹⁰ or a radical of the formula 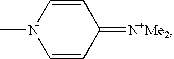
   if appropriate in the presence of a catalyst and in the presence of a diluent, or
g) hydroxypyrazolopyrimidines of the formula (X) 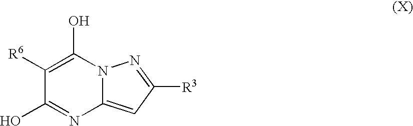
in which
   R³ and R⁶ are as defined above are reacted with phosphorus oxychloride in the presence of dimethylformamide and, if appropriate, allowed to react further with addition of phosphorus pentachloride, and the resulting halopyrazolopyrimidines of the formula (XI) 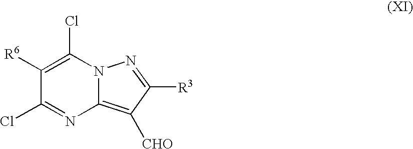
   in which
   R³ and R⁶ are as defined above are reacted with amines of the formula (XII) 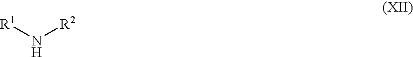
   in which
   R¹ and R² are as defined above, if appropriate in the presence of a catalyst, if appropriate in the presence of an acid binder and if appropriate in the presence of a diluent.

The cyano compounds of the formula (VI) are obtained when
h) halopyrazolopyrimidines of the formula 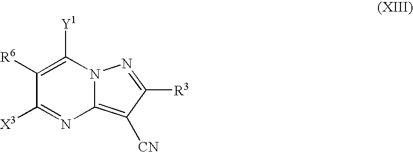
in which
   R³ and R⁶ are as defined above,
   X³ represents halogen and
   Y¹ represents halogen are reacted with amines of the formula 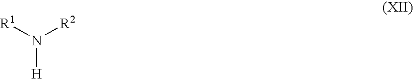
   in which
   R¹ and R² are as defined above, if appropriate in the presence of a diluent, if appropriate in the presence of a catalyst and if appropriate in the presence of an acid acceptor, and, if appropriate, the resulting cyano compounds of the formula 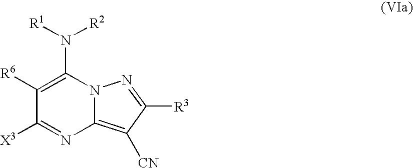
   in which
   R¹, R², R³, R⁶ and X³ are as defined above are, in a second step, reacted with alcohols or mercaptans of the formula
      R¹¹-Z-H (XIV)
   in which
   R¹¹ represents alkyl and
   Z represents oxygen or sulfur in the presence of a base and, if appropriate, in the presence of a diluent.

The halopyrazolopyrimidines of the formula (XIII) are known or can be prepared by known methods (cf. DE-A 103 28 996 and PCT/EP 03/05 159).

Thus, halopyrazolopyrimidines of the formula (XIII) are obtained when
i) dihydroxypyrazolopyrimidines of the formula 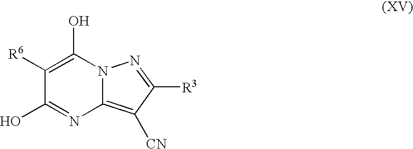
in which
   R³ and R⁶ are as defined above are reacted with halogenating agents, if appropriate in the presence of a diluent.

The dihydroxypyrazolopyrimidines of the formula (XV) are obtained when
j) malonic esters of the formula 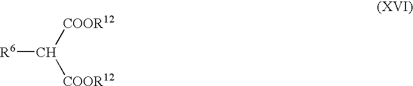
in which
   R⁶ is as defined above and
   R¹² represents alkyl are reacted with aminopyrazoles of the formula 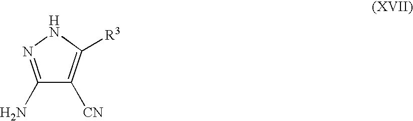
   in which
   R³ is as defined above, if appropriate in the presence of a diluent and if appropriate in the presence of a strong base.

The formula (XVI) provides a general definition of the malonic esters required as starting materials for carrying out the process (j). In this formula, R⁶ preferably has those meanings which have already been mentioned in connection with the description of the compounds of the formula (I) according to the invention as being preferred for this radical. R¹² preferably represents alkyl having 1 to 4 carbon atoms, particularly preferably methyl or ethyl.

The malonic esters of the formula (XVI) are known or can be prepared by known methods (cf. U.S. Pat. No. 6,156,925).

Suitable diluents for carrying out the process (j) are all customary inert organic solvents. Preference is given to using aliphatic, alicyclic or aromatic hydrocarbons, such as petroleum ether, hexane, heptane, cyclohexane, methylcyclohexane, benzene, toluene, xylene or decalin; halogenated hydrocarbons, such as chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, or 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulfoxides, such as dimethyl sulfoxide; sulfones, such as sulfolane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether; amines, such as tri-n-butylamine, or carboxylic acids, such as acetic acid.

Suitable strong bases for carrying out the process (j) are, preferably, alkaline earth metal or alkali metal hydrides or alkoxides, and also alkali metal amides. Sodium hydride, sodium amide, sodium methoxide, sodium ethoxide and potassium tert-butoxide may be mentioned by way of example.

Furthermore suitable are tertiary amines, such as tri-n-butylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU). If the bases are liquid substances, they may simultaneously act as diluent.

Process (j) and also the other processes described in the present patent application are generally carried out under atmospheric pressure. However, it is also possible to operate under elevated pressure or—as long as no highly volatile reaction components are present—under reduced pressure.

When carrying out the process (j), the reaction temperatures may in each case be varied within a relatively wide range. If bases are absent, the process is generally carried out at temperatures between 100° C. and 250° C., preferably between 120° C. and 200° C. If bases are present, the process is generally carried out at temperatures between 20° C. and 120° C., preferably between 20° C. and 80° C.

When carrying out the process (j), in general from 1 to 15 mol, preferably from 1 to 8 mol, of aminopyrazole of the formula (XVII) are employed per mole of malonic ester of the formula (XVI). Work-up is carried out by customary methods.

Suitable halogenating agents for carrying out the process (i) are all customary reagents suitable for exchanging hydroxyl groups attached to carbon for halogen. Preference is given to using phosphorus trichloride, phosphorus tribromide, phosphorus pentachloride, phosphorus oxychloride, phosgene, thionyl chloride, thionyl bromide or mixtures thereof. The corresponding fluorine compounds of the formula (XIII) can be prepared from the chlorine or bromine compounds by reaction with potassium fluoride.

Suitable diluents for carrying out the process (i) are all organic solvents customary for such halogenations. Preference is given to using aliphatic, alicyclic or aromatic hydrocarbons, such as petroleum ether, hexane, heptane, cyclohexane, methylcyclohexane, benzene, toluene, xylene or decalin; halogenated hydrocarbons, such as chlorobenzene, dichlorobenzene, dichloromethane, chloroform, carbon tetrachloride, dichloroethane or trichloroethane.

However, it is also possible for the halogenating agent itself or a mixture of halogenating agent and one of the diluents mentioned to serve as diluent.

When carrying out the process (i), the reaction temperatures can be varied within a relatively wide range. In general, the process is carried out at temperatures between 20° C. and 150° C., preferably between 40° C. and 120° C.

When carrying out the process (i), in each case an excess of halogenating agent is employed per mole of dihydroxypyrazolopyrimidine of the formula (XV). Work-up is carried out by customary methods.

The formula (XIII) provides a general definition of the halopyrazolopyrimidines required as starting materials for carrying out the process (h). In this formula, R³ and R⁶ preferably have those meanings which have already been mentioned in connection with the description of the compounds of the formula (I) according to the invention as being preferred for these radicals. X³ and Y¹ each preferably represent fluorine, chlorine or bromine, particularly preferably fluorine or chlorine.

The formula (XII) provides a general definition of the amines required as reaction components for carrying out the process (h). In this formula, R¹ and R² preferably have those meanings which have already been mentioned in connection with the description of the compounds of the formula (I) according to the invention as being preferred for these radicals.

The formula (XIV) provides a general definition of the alcohols and mercaptans required as reaction components in the second step of process (h). In this formula, R¹¹ preferably represents alkyl having 1 to 4 carbon atoms, particularly preferably methyl or ethyl. Z also preferably represents an oxygen or a sulfur atom.

Suitable diluents for carrying out the first step of the process (h) are all customary inert organic solvents. Preference is given to using aliphatic, alicyclic or aromatic hydrocarbons, such as petroleum ether, hexane, heptane, cyclohexane, methylcyclohexane, benzene, toluene, xylene or decalin; halogenated hydrocarbons, such as chlorobenzene, dichlorobenzene, dichloromethane, chloroform, carbon tetrachloride, dichloroethane or trichloroethane; ethers, such as diethyl ether, diisopropyl ether, methyl t-butyl ether, dioxane, tetrahydrofuran, 1,2-dimethoxyethane or 1,2-diethoxyethane; amides, such as N,N-dimethylformamide, N,N-dimethylacetamide or N-methyl-pyrrolidone; esters, such as methyl acetate or ethyl acetate; sulfoxides, such as dimethyl sulfoxide; sulfones, such as sulfolane.

Suitable catalysts for carrying out the first step of the process (h) are all reaction promoters customary for such reactions. Preference is given to using alkali metal fluorides, such as potassium fluoride or cesium fluoride.

Suitable acid acceptors for carrying out the first step of the process (h) are all acid binders customary for such reactions. Preference is given to using ammonia and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the first step of the process (h), the reaction temperatures can be varied within a relatively wide range. In general, the process is carried out at temperatures between 0° C. and 150° C., preferably at temperatures between 0° C. and 80° C.

When carrying out the first step of the process (h), in general from 0.5 to 10 mol, preferably from 0.8 to 2 mol, of amine of the formula (XII) are employed per mole of halopyrazolopyrimidine of the formula (XIII). Work-up is carried out by customary methods.

Suitable bases for carrying out the second step of the process (h) are all inorganic and organic acid binders customary for such reactions. Preference is given to using alkali metal hydroxides and carbonates, such as sodium hydroxide, potassium hydroxide, sodium carbonate or potassium carbonate, furthermore alkali metal alkoxides, such as sodium methoxide or potassium tert-butoxide, and furthermore tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

Suitable diluents for carrying out the second step of the process (h) are all inert organic solvents customary for such reactions. Preferably, an excess of the alcohol or mercaptan of the formula (XIV) acting as reaction component simultaneously serves as diluent.

When carrying out the second step of the process (h), the reaction temperatures can be varied within a relatively wide range. In general, the process is carried out at temperatures between 0° C. and 150° C., preferably between 20° C. and 120° C.

When carrying out the second step of the process (h), preferably from 2 to 3 mol or else a larger excess of alcohol or mercaptan of the formula (XIV) and 2 to 3 equivalents of base are employed per mole of cyano compound of the formula (VIa). Work-up is carried out by customary methods.

Formula (VII) provides a general definition of the Grignard compounds required as reaction components for carrying out the process (e, variant β). In this formula, R¹⁰ preferably represents alkyl having 1 to 4 carbon atoms, particularly preferably methyl, ethyl, n-propyl or n-butyl. X¹ also preferably represents chlorine or bromine.

The Grignard compounds of the formula (VII) are known or can be prepared by known methods.

Suitable diluents for carrying out the process (e, variant α) are all customary inert organic solvents. Preference is given to using aliphatic or aromatic, optionally halogenated, hydrocarbons, such as toluene, dichloromethane, chloroform or carbon tetrachloride.

When carrying out the process (e, variant α), the reaction temperatures can be varied within a certain range. In general, the process is carried out at temperatures between −80° C. and +20° C., preferably between −60° C. and +10° C.

When carrying out the process (e, variant α), in general an equivalent amount or else an excess, preferably from 1.1 to 1.2 mol, of diisobutylaluminum hydride is employed per mole of cyano compound of the formula (VI), and an excess of aqueous ammonium chloride solution is then added. Work-up is carried out by customary methods. In general, the reaction mixture is acidified, the organic phase is removed, the aqueous phase is extracted with a poorly water-miscible organic solvent and the combined organic phases are washed, dried and concentrated under reduced pressure.

Suitable catalysts for carrying out the process (e, variant β) are all reaction promoters customary for such Grignard reactions. Potassium iodide and iodine may be mentioned by way of example.

Suitable diluents for carrying out the process (e, variant β) are all inert organic solvents customary for such reactions. Preference is given to using ethers, such as diethyl ether, dioxane or tetrahydrofuran, moreover aromatic hydrocarbons, such as toluene, and also mixtures of ethers and aromatic hydrocarbons, such as toluene/tetrahydrofuran.

When carrying out the process (e, variant β), the reaction temperatures can be varied within a certain range. In general, the process is carried out at temperatures between −20° C. and +100° C., preferably between 0° C. and 80° C.

When carrying out the process (e, variant β), in general from 2 to 3 mol of Grignard compound of the formula (VII) are employed per mole of cyano compound of the formula (VI). This is followed by an aqueous work-up according to customary methods.

The formula (VIII) provides a general definition of the pyrazolopyrimidines required as starting materials for carrying out the process (f). In this formula, R¹, R², R³, R⁵ and R⁶ preferably have those meanings which have already been mentioned in connection with the description of the compounds of the formula (I) according to the invention as being preferred for these radicals.

The pyrazolopyrimidines of the formula (VIII) are known or can be prepared by known methods.

The formula (IX) provides a general definition of the acid halides and acid anhydrides required as reaction components for carrying out the process (f). In this formula, R¹⁰ preferably represents alkyl having 1 to 4 carbon atoms, particularly preferably methyl or ethyl. X² preferably represents chlorine or bromine and also represents a radical of the formula —O—CO—R¹⁰, where R¹⁰ represents alkyl having 1 to 4 carbon atoms, particularly preferably methyl or ethyl.

The carboxylic acid derivatives of the formula (IX) are known.

Suitable catalysts for carrying out the process (f) are all reaction promoters customarily used for Friedel-Crafts reactions. Preference is given to using Lewis acids, such as aluminum trichloride, aluminum tribromide and iron(III) chloride.

Suitable diluents for carrying out the process (f) are all inert organic solvents customary for such Friedel-Crafts reactions. Preference is given to using ethers, such as diethyl ether, methyl tert-butyl ether, dioxane and tetrahydrofuran, and also carbon disulfide.

When carrying out the process (f), the reaction temperatures can be varied within a certain range. In general, the process is carried out at temperatures between −10° C. and +100° C., preferably between 0° C. and 60° C.

When carrying out the process (f), in general from 1 to 5 mol of acid halide of the formula (IX) and from 1.1 to 5 mol, preferably from 1.1 to 3 mol, of catalyst are employed per mole of pyrazolopyrimidine of the formula (VIII). If acid anhydrides are used as reaction components, in general from 1 to 2 mol of acid anhydride of the formula (IX) and from 2.1 to 6 mol, preferably from 2.1 to 4 mol, of catalyst are employed per mole of pyrazolopyrimidine of formula (VIII). In general, the reaction components are initially added at low temperature and, after the initially vigorous reaction has subsided, the mixture is slowly heated to reflux temperature. Work-up is carried out by customary methods.

The formula (X) provides a general definition of the hydroxypyrazolopyrimidines required as starting materials for carrying out the process (g). In this formula, R³ and R⁶ preferably have those meanings which have already been mentioned in connection with the description of the compounds of the formula (I) according to the invention as being preferred for these radicals.

The hydroxypyrazolopyrimidines of the formula (X) can be prepared by the process (j) if aminopyrazoles of the formula (XVII) are used which, instead of the CN group, carry a hydrogen atom.

The first step of the process (g) is carried out under the conditions of the Vilsmeier formulation using phosphorus oxychloride in the presence of dimethylformamide. Here, it is also possible to add phosphorus pentachloride as chlorinating agent.

When carrying out the first step of the process (g), the reaction temperatures can be varied within a relatively wide range. In general, the process is carried out at temperatures between −10° C. and +150° C., preferably between 0° C. and 120° C.

When carrying out the first step of the process (g), in general from 2 to 5 mol of dimethylformamide, from 5 to 15 mol of phosphorus oxychloride and, if appropriate, from 0 to 2 mol of phosphorus pentachloride are employed per mole of hydroxypyrazolopyrimidine of the formula (X). Work-up is carried out by customary methods.

Suitable for carrying out the second step of the process (g) are the amines of the formula (XII) and those catalysts, acid binders and diluents which have already been mentioned in connection with the description of the process (h). Reaction temperatures and the other reaction conditions also correspond to those used in the case of process (h).

The formula (III) provides a general definition of the triphenylphosphonium bromides required as reaction components for carrying out the process (a) according to the invention. In this formula, Ph represents phenyl. R⁸ preferably represents hydrogen or alkyl having 1 to 4 carbon atoms, where the alkyl radicals may be substituted by carboxyl, methoxycarbonyl, ethoxycarbonyl or halogen. Particularly preferably, R⁸ represents hydrogen, methyl or ethyl, where the two last-mentioned radicals may be substituted by carboxyl, methoxycarbonyl or ethoxycarbonyl.

The triphenylphosphonium bromides of the formula (III) are known or can be prepared by known methods.

Suitable bases for carrying out the process (a) according to the invention are all deprotonating agents customary for such Wittig reactions. Preference is given to using butyllithium.

Suitable diluents for carrying out the process (a) according to the invention are all organic solvents customary for such Wittig reactions. Preference is given to using ethers, such as dioxane or tetrahydrofuran.

When carrying out the process (a) according to the invention, the reaction temperatures can be varied within a certain range. In general, the process is carried out at temperatures between −78° C. and +30° C.

When carrying out the process (a) according to the invention, an equivalent amount or else an excess of triphenylphosphonium bromide of the formula (III) and an equivalent amount or else an excess of base are employed per mole of pyrazolopyrimidine of the formula (II). Work-up is carried out by customary methods.

The formula (IV) provides a general definition of the pyrazolopyrimidines required as starting materials for carrying out the process (b) according to the invention. In this formula, R¹, R², R³, R⁵ and R⁶ preferably have those meanings which have already been mentioned in connection with the description of the compounds of the formula (I) according to the invention as being preferred for these radicals. R⁹ preferably represents hydrogen or alkyl having 1 to 4 carbon atoms, where each of the alkyl radicals may be monosubstituted by carboxyl, methoxycarbonyl or ethoxycarbonyl. Particularly preferably, R⁹ represents hydrogen, methyl, ethyl or propyl, where the three last-mentioned radicals may each be monosubstituted by carbonyl, methoxycarbonyl or ethoxycarbonyl. X also preferably represents chlorine or bromine.

The pyrazolopyrimidines of the formula (IV) are obtained when
k) pyrazolopyrimidines of the formula 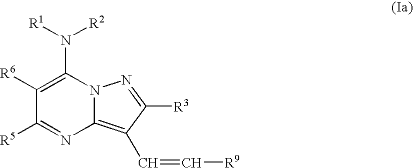
in which
   R¹, R², R³, R⁵, R⁶ and R⁹ are as defined above are reacted with bromine or chlorine in the presence of an inert organic diluent, such as dichloromethane, trichloromethane or carbon tetrachloride, at temperatures between −20° C. and +20° C. Here, the reaction components are preferably employed in approximately equivalent amounts. Work-up is carried out by customary methods.

Suitable strong bases for carrying out the process (b) according to the invention are, preferably, alkali metal alkoxides, where sodium methoxide and potassium tert-butoxide may be mentioned by way of example. Furthermore suitable are tertiary amines as already mentioned in connection with the description of the process (h).

Suitable diluents for carrying out the process (b) according to the invention are all inert organic solvents customary for such reactions. Preference is given to using alcohols, such as methanol or ethanol, and also nitriles, such as acetonitrile.

When carrying out process (b) according to the invention, the temperatures can be varied within a certain range. In general, the process is carried out at temperatures between −10° C. and +80° C., preferably between 0° C. and 60° C.

When carrying out the process (b) according to the invention, in general from 2 to 3 equivalents or else a larger excess of strong base is employed per mole of pyrazolopyrimidine of the formula (IV). Work-up is again carried out by customary methods.

The formula (IIa) provides a general definition of the pyrazolopyrimidines required as starting materials for carrying out the process (c) according to the invention. In this formula, R¹, R², R³, R⁵, R⁶ and R⁹ preferably have those meanings which have already been mentioned above as being preferred for these radicals.

The pyrazolopyrimidines of the formula (IIa) can be prepared by the process (e) or (f) already described.

When carrying out the first step of the process (c) according to the invention, the temperatures can be varied within a relatively wide range. In general, the process is carried out at temperatures between −10° C. and +150° C., preferably between 0° C. and 120° C.

When carrying out the first step of the process (c) according to the invention, in general from 2 to 5 mol of dimethylformamide and from 3 to 5 mol of phosphorus oxychloride are employed per mole of pyrazolopyrimidine of the formula (IIa). Work-up is carried out by customary methods.

Suitable bases and diluents for the further practice of the process (c) according to the invention are all those components which have already been mentioned in connection with the description of the process (h) as being suitable for this purpose.

In the further practice of the process (c) according to the invention, the reaction temperatures can be varied within a relatively wide range. In general, the process is carried out at temperatures between 0° C. and 150° C., preferably between 20° C. and 120° C.

When carrying out the second step of the process (c) according to the invention, in general an equivalent amount or else an excess of base is employed per mole of the compound of the formula (V). Work-up is again carried out by customary methods.

The processes described above are generally carried out under atmospheric pressure. However, it is also possible to operate under elevated pressure.

The compounds according to the invention have potent microbicidal activity and can be employed for controlling unwanted microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:

Xanthomonas species, such as, for example, Xanthomonas campestris pv. oryzae;

Pseudomonas species, such as, for example, Pseudomonas syringae pv. lachrymans;

Erwinia species, such as, for example, Erwinia amylovora;

Pythium species, such as, for example, Pythium ultimum;

Phytophthora species, such as, for example, Phytophthora infestans;

Pseudoperonospora species, such as, for example, Pseudoperonospora humuli or Pseudoperonospora cubensis;

Plasmopara species, such as, for example, Plasmopara viticola;

Bremia species, such as, for example, Bremia lactucae;

Peronospora species, such as, for example, Peronospora pisi or P. brassicae;

Erysiphe species, such as, for example, Erysiphe graminis;

Sphaerotheca species, such as, for example, Sphaerotheca fuliginea;

Podosphaera species, such as, for example, Podosphaera leucotricha;

Venturia species, such as, for example, Venturia inaequalis;

Pyrenophora species, such as, for example, Pyrenophora teres or P. graminea (conidia form: Drechslera, syn: Helminthosporium);

Cochliobolus species, such as, for example, Cochliobolus sativus (conidia form: Drechslera, syn: Helminthosporium);

Uromyces species, such as, for example, Uromyces appendiculatus;

Puccinia species, such as, for example, Puccinia recondita;

Sclerotinia species, such as, for example, Sclerotinia sclerotiorum;

Tilletia species, such as, for example, Tilletia caries;

Ustilago species, such as, for example, Ustilago nuda or Ustilago avenae;

Pellicularia species, such as, for example, Pellicularia sasakii;

Pyricularia species, such as, for example, Pyricularia oryzae;

Fusarium species, such as, for example, Fusarium culmorum;

Botrytis species, such as, for example, Botrytis cinerea;

Septoria species, such as, for example, Septoria nodorum;

Leptosphaeria species, such as, for example, Leptosphaeria nodorum;

Cercospora species, such as, for example, Cercospora canescens;

Alternaria species, such as, for example, Alternaria brassicae; and

Pseudocercosporella species, such as, for example, Pseudocercosporella herpotrichoides.

The active compounds according to the invention also show a strong invigorating action in plants. Accordingly, they are suitable for mobilizing the internal defenses of the plant against attack by unwanted microorganisms.

In the present context, plant-invigorating (resistance-inducing) compounds are to be understood as meaning substances which are capable of stimulating the defense system of plants such that, when the treated plants are subsequently inoculated with unwanted microorganisms, they display substantial resistance to these microorganisms.

In the present case, unwanted microorganisms are to be understood as meaning phytopathogenic fungi, bacteria and viruses. The compounds according to the invention can thus be used to protect plants within a certain period of time after treatment against attack by the pathogens mentioned. The period of time for which this protection is achieved is generally from 1 to 10 days, preferably 1 to 7 days, from the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be employed with particularly good results for controlling cereal diseases, such as, for example, against Erysiphe species, and diseases in viticulture and in the cultivation of fruit and vegetables, such as, for example, against Botrytis, Venturia, Sphaerotheca and Podosphaera species.

The active compounds according to the invention are also suitable for increasing the yield of crops. In addition, they show reduced toxicity and are well tolerated by plants.

If appropriate, the active compounds according to the invention can, at certain concentrations and application rates, also be employed as herbicides, for regulating plant growth and for controlling animal pests. If appropriate, they can also be used as intermediates or precursors in the synthesis of other active compounds.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested material and vegetative and generative propagation material, for example seedlings, tubers, rhizomes, cuttings and seeds.

The treatment of the plants and parts of plants according to the invention with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multilayer coating.

In the protection of materials, the compounds according to the invention can be employed for protecting industrial materials against infection with, and destruction by, unwanted microorganisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular molds, wood-discoloring and wood-destroying fungi (Basidiomycetes) and against slime organisms and algae.

Microorganisms of the following genera may be mentioned as examples:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa, and

Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulfoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, pumice, marble, sepiolite and dolomite, or else synthetic granules of inorganic and organic meals, and granules of organic material such as sawdust, coconut shells, corn cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates, or else protein hydrolysates. Suitable dispersants are: for example lignosulfite waste liquors and methylcellulose.

Tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can, as such or in their formulations, also be used in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Suitable mixing components are, for example, the following compounds:

Fungicides:

2-phenylphenol; 8-hydroxyquinoline sulfate; acibenzolar-S-methyl; aldimorph; amidoflumet; ampropylfos; ampropylfos-potassium; andoprim; anilazine; azaconazole; azoxystrobin; benalaxyl; benalaxyl-M; benodanil; benomyl; benthiavalicarb-isopropyl; benzamacril; benzamacril-isobutyl; bilanafos; binapacryl; biphenyl; bitertanol; blasticidin-S; boscalid; bromuconazole; bupirimate; buthiobate; butylamine; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propynyl]oxy]-3-methoxyphenyl]ethyl]-3-methyl-2-[(methylsulfonyl)amino]butanamide; 1-(1-naphthalenyl)-1H-pyrrole-2,5-dione; 2,3,5,6-tetrachloro-4-(methylsulfonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridine-dicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropanecarboxamide; N-butyl-8-(1,1-dimethylethyl)-1-oxa-spiro[4.5]decane-3-amine; sodium tetracarbonate; and copper salts and preparations, such as Bordeaux mixture; copper hydroxide; copper naphthenate; copper oxychloride; copper sulfate; cufraneb; copper oxide; mancopper; oxine-copper.

Bactericides:

bronopol, dichlorophen, nitrapyrin, nickel dimethyldithiocarbamate, kasugarnycin, octhilinone, furancarboxylic acid, oxytetracyclin, probenazole, streptomycin, tecloftalam, copper sulfate and other copper preparations.

Insecticides/Acaricides/Nematicides:

1. Acetylcholinesterase (AChE) inhibitors
   1.1 carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)
   1.2 organophosphates (for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulfone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)
2. Sodium channel modulators/blockers of voltage-gated sodium channels
   2.1 pyrethroids (for example acrinathrin, allethrin (d-cis-trans, d-trans), beta-cyfluthrin, bifenthrin, bioallethrin, bioallethrin-S-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (IR-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R-isomer), tralomethrin, transfluthrin, ZXI 8901, pyrethrins (pyrethrum))
   2.2 oxadiazines (for example indoxacarb)
3. Acetylcholine receptor agonists/antagonists
   3.1 chloronicotinyls/neonicotinoids (for example acetamiprid, clothianidin, dinotefuran, imidacloprid, nitenpyram, nithiazine, thiacloprid, thiamethoxam)
   3.2 nicotine, bensultap, cartap
4. Acetylcholine receptor modulators
   4.1 spinosyns (for example spinosad)
5. Antagonists of GABA-gated chloride channels
   5.1 cyclodiene organochlorines (for example camphechlor, chlordane, endosulfan, gamma-HCH, HCH, heptachlor, lindane, methoxychlor
   5.2 fiproles (for example acetoprole, ethiprole, fipronil, vaniliprole)
6. Chloride channel activators
   6.1 mectins (for example abamectin, avermectin, emamectin, emamectin-benzoate, ivermectin, milbemectin, milbemycin)
7. Juvenile hormone mimetics
   (for example diofenolan, epofenonane, fenoxycarb, hydroprene, kinoprene, methoprene, pyriproxifen, triprene)
8. Ecdyson agonists/disruptors
   8.1 diacylhydrazines (for example chromafenozide, halofenozide, methoxyfenozide, tebufenozide)
9. Chitin biosynthesis inhibitors
   9.1 benzoylureas (for example bistrifluron, chlofluazuron, diflubenzuron, fluazuron, flucycloxuron, flufenoxuron, hexaflumuron, lufenuron, novaluron, noviflumuron, penfluron, teflubenzuron, triflumuron)
   9.2 buprofezin
   9.3 cyromazine
10. Inhibitors of oxidative phosphorylation, ATP disruptors
   10.1 diafenthiuron
   10.2 organotins (for example azocyclotin, cyhexatin, fenbutatin-oxide)
11. Decouplers of oxidative phosphorylation acting by interrupting the h-proton gradient
   11.1 pyrroles (for example chlorfenapyr)
   11.2 dinitrophenols (for example binapacryl, dinobuton, dinocap, DNOC)
12. Site-I electron transport inhibitors
   12.1 METIs (for example fenazaquin, fenpyroximate, pyrimidifen, pyridaben, tebufenpyrad, tolfenpyrad)
   12.2 hydramethylnone
   12.3 dicofol
13. Site-II electron transport inhibitors
   13.1 rotenone
14. Site-III electron transport inhibitors
   14.1 acequinocyl, fluacrypyrim
15. Microbial disruptors of the insect gut membrane
   Bacillus thuringiensis strains
16 Inhibitors of fat synthesis
   16.1 tetronic acids (for example spirodiclofen, spiromesifen)
   16.2 tetramic acids [for example 3-(2,5-dimethylphenyl)-8-methoxy-2-oxo-1-azaspiro[4.5]dec-3-en4-yl ethyl carbonate (alias: carbonic acid, 3-(2,5-dimethylphenyl)-8-methoxy-2-oxo-1-azaspiro[4.5]dec-3-en4-yl ethyl ester, CAS Reg. No.: 382608-10-8) and carbonic acid, cis-3-(2,5-dimethylphenyl)-8-methoxy-2-oxo-1-azaspiro[4.5]dec-3-en4-yl ethyl ester (CAS Reg. No.: 203313-25-1)]
17. Carboxamides
   (for example flonicamid)
18. Octopaminergic agonists
   (for example amitraz)
19. Inhibitors of magnesium-stimulated ATPase
   (for example propargite)
20. Phthalamides
   (for example N²-[1,1-dimethyl-2-(methylsulfonyl)ethyl]-3-iodo-N¹-[2-methyl-4-[1,2,2,2-tetrafluoro-1-(trifluoromethyl)ethyl]phenyl]-1,2-benzenedicarboxamide (CAS Reg. No.: 272451-65-7), flubendiamide)
21. Nereistoxin analogs
   (for example thiocyclam hydrogen oxalate, thiosultap-sodium)
22. Biologicals, hormones or pheromones
   (for example azadirachtin, Bacillus spec., Beauveria spec., codlemone, Metarrhizium spec., Paecilomyces spec., thuringiensin, Verticillium spec.)
23. Active compounds with unknown or unspecific mechanisms of action
   23.1 fumigants (for example aluminum phosphide, methyl bromide, sulfuryl fluoride)
   23.2 selective antifeedants (for example cryolite, flonicamid, pymetrozine)
   23.3 mite growth inhibitors (for example clofentezine, etoxazole, hexythiazox)
   23.4 amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin, furthermore the compound 3-methylphenyl propylcarbamate (Tsumacide Z), the compound 3-(5-chloro-3-pyridinyl)-8-2,2,2-trifluoroethyl)-8-azabicyclo[3.2.1]octane-3-carbonitrile (CAS Reg. No. 185982-80-3) and the corresponding 3-endo-isomer (CAS Reg. No. 185984-60-5) (cf. WO 96/37494, WO 98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

In addition, the compounds of the formula (I) according to the invention also have very good antimycotic activity. They have a very broad antimycotic activity spectrum in particular against dermatophytes and yeasts, molds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The list of these fungi does by no means limit the mycotic spectrum which can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10 000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10 000 g/ha, preferably between 1 and 5 000 g/ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof, are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), corn, soybeans, potatoes, cotton, tobacco, oilseed rape and also fruit plants (with the fruits apples, pears, citrus fruits and grapes), and particular emphasis is given to corn, soybeans, potatoes, cotton, tobacco and oilseed rape. Traits that are particularly emphasized are increased defense of the plants against insects, arachnids, nematodes and slugs and snails by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defense of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulfonylureas, glyphosate or phosphinotricin (for example the “PAT” gene). The genes which impart the desired traits in question can also be present in combination with one another in the transgenic plants. Examples of “Bt plants” which may be mentioned are corn varieties, cotton varieties, soybean varieties and potato varieties which are sold under the trade names YIELD GARD® (for example corn, cotton, soybeans), KnockOut® (for example corn), StarLink® (for example corn), Bollgard® (cotton), Nucoton® (cotton) and NewLeaf® (potato). Examples of herbicide-tolerant plants which may be mentioned are corn varieties, cotton varieties and soybean varieties which are sold under the trade names Roundup Ready® (tolerance to glyphosate, for example corn, cotton, soybean), Liberty Link® (tolerance to phosphinotricin, for example oilseed rape), IMI® (tolerance to imidazolinones) and STS® (tolerance to sulfonylureas, for example corn). Herbicide-resistant plants (plants bred in a conventional manner for herbicide tolerance) which may be mentioned also include the varieties sold under the name Clearfield® (for example corn). Of course, these statements also apply to plant cultivars which have these genetic traits or genetic traits still to be developed, and which will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The compounds of the formula (I) according to the invention are furthermore suitable for suppressing the growth of tumour cells in humans and mammals. This is based on an interaction of the compounds according to the invention with tubulin and microtubuli and by promoting microtubuli polymerization.

For this purpose, it is possible to administer an effective amount of one or more compounds of the formula (I) or pharmaceutically acceptable salts thereof.

The preparation and the use of the active compounds according to the invention are illustrated in the examples below.

### PREPARATION EXAMPLES

### Example 1

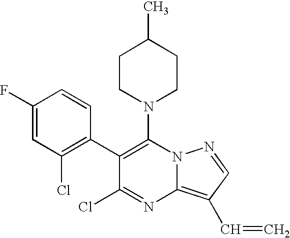

Process (a):

At −70° C., 0.173 g (2.701 mmol) of n-butyllithium (as a 2.5 molar solution in hexane) is added with stirring to a solution of 0.965 g (2.701 mmol) of triphenylmethylphosphonium bromide in 58 ml of tetrahydrofuran. The mixture is stirred at −70° C. for 15 minutes, and, at the same temperature, 1.0 g (2.455 mmol) of 3-formyl-5-chloro-6-(2-chloro-4-fluorophenyl)-7-(4-methylpiperidino)pyrazolo-[1,5a]pyrimidine is then added with stirring. The reaction mixture is subsequently stirred at room temperature for 16 hours and then concentrated under reduced pressure. The residue that remains is triturated with water and ethyl acetate. The resulting mixture is extracted three times with ethyl acetate and the combined organic phases are dried with sodium sulfate and then concentrated under reduced pressure. The residue that remains is chromatographed on silica gel using a mixture of 4 parts of cyclohexane and 1 part of ethyl acetate. This gives 0.24 g (19% of theory) of 3-vinyl-5-chloro-6-(2-chloro-4-fluorophenyl)-7-(4-methylpiperidino)pyrazolo[1 ,5a]pyrimidine.

HPLC: log P=6.07

### Example 9

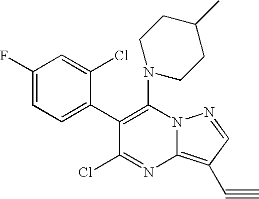

Process (a):

4.283 g (9.821 mmol) of bromomethyltriphenylphosphonium bromide are initially charged in 100 ml of dioxane, and 1.102 g (9.821 mmol) of potassium tert-butoxide are added at from 0 to 10° C. The mixture is stirred for another 15 min, 2.000 g (4.911 mmol) of 3-formyl-5-chloro-6-(2-chloro-4-fluorophenyl)-7-(4-methylpiperidino)pyrazolo[1,5a]pyrimidine are then added and the mixture is stirred at room temperature overnight. The mixture is concentrated, triturated with water and ethyl acetate and extracted, and the organic phase is dried. The residue is chromatographed on silica gel using cyclohexane/ethyl acetate (4:1).

HPLC: logP=5.31

### Example 10

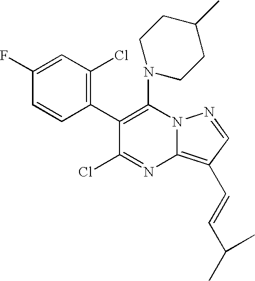

Process (d):

1.000 g (2,455 mmol) of 3-formyl-5-chloro-6-(2-chloro-4-fluorophenyl)-7-(4-methylpiperidino)-pyrazolo[1,5a]pyrimidine is initially charged in 50 ml of tetrahydrofuran at 0° C., 0.436 g (2.701 mmol) of 2-methylpropylmagnesium bromide is added a little at a time and the mixture is stirred at room temperature overnight. The mixture is concentrated and triturated with water/methylene chloride, hydrochloric acid is added, the mixture is extracted and the organic phase is dried. After concentration, the residue is chromatographed on silica gel using cyclohexane/ethyl acetate 4:1.

HPLC: log P=7.21

### Example 29

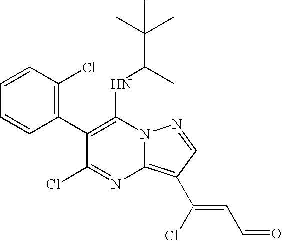

Process (c):

At 0° C., 0.400 g (0.987 mmol) of 3-methylcarbonyl-5-chloro-6-(2-chlorophenyl)-7-(3,3-dimethylbut-2-ylamino)pyrazolo[1,5a]pyrimidine is initially charged in 51.5 g of dimethylformamide. 0.378 g (2.467 mol) of phosphoryl chloride is added dropwise, and the mixture is allowed to warm to room temperature and stirred for another 12 h. The mixture is concentrated, dioxane and then 10% strength NaOH are added and the mixture is stirred at room temperature for 24 h. The mixture is concentrated, the residue is taken up in water/ethyl acetate and extracted and the organic phase is dried and concentrated. Trituration with ethyl acetate and drying on clay gives the product as a light-yellow solid.

HPLC: log P=5.98

The compounds listed in the tables below are likewise prepared by the methods described above.

TABLE 1

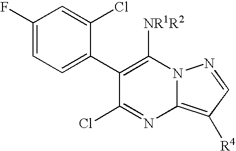

| Ex. No. | 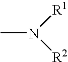 | R⁴ | logP (pH 2.3) |
|---|---|---|---|
| 1 | 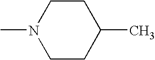 | —CH═CH₂ | 6.07 |
| 2 | 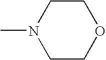 | —CH═CH₂ | 4.34 |
| 3 | 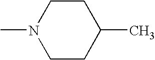 | —CH═CH—COOH | 4.50 |
| 4 | 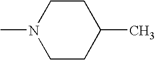 | —CH═CH—COOC₂H₅ | 6.05 |
| 5 | 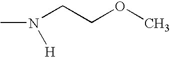 | —CH═CH₂ | 4.27 |
| 6 | 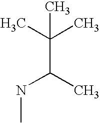 | —CH═CH₂ | 6.00 |
| 7 | 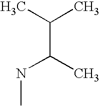 | —CH═CH₂ | 5.60 |
| 8 | 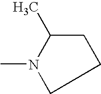 | —CH═CH₂ | 5.45 |
| 9 | 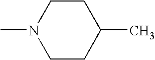 | —C≡CH | 5.31 |
| 10 | 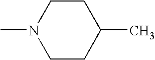 | —CH═CH—CH(CH₃)₂ | 7.21 |
| 11 | 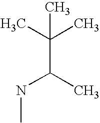 | —C(Cl)═CH—CHO | |
| 12 | 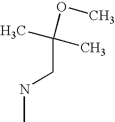 | —CH═CH₂ | 5.01 |
| 13 | 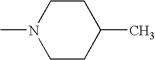 | —C(Cl)═CH—CHO | 6.06 |
| 14 | 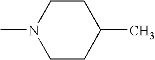 | —C(CH₃)═CH2 | 6.62 |
| 15 | 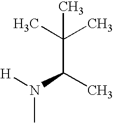 | —CH═CH—Br | 6.64 |
| 16 | 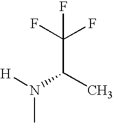 | —CH═CH₂ | 4.99 |
| 17 | 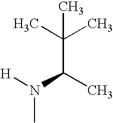 | —C≡CH | 5.30 |
| 18 | 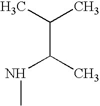 | —C≡CH | 4.87 |

TABLE 2

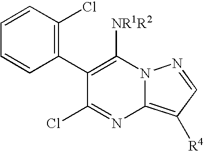

| Ex. No. | 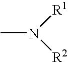 | R⁴ | logP (pH 2.3) |
|---|---|---|---|
| 19 | 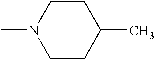 | —CH═CH₂ | |
| 20 | 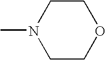 | —CH═CH₂ | |
| 21 | 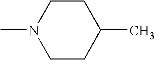 | —CH═CH—COOH | |
| 22 | 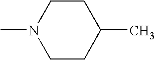 | —CH═CH—COOC₂H₅ | |
| 23 | 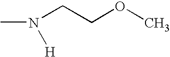 | —CH═CH₂ | |
| 24 | 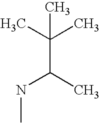 | —CH═CH₂ | |
| 25 | 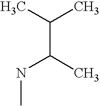 | —CH═CH₂ | |
| 26 | 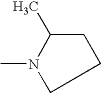 | —CH═CH₂ | |
| 27 | 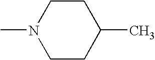 | —C≡CH | |
| 28 | 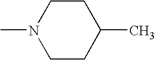 | —CH═CH—CH(CH₃)₂ | |
| 29 | 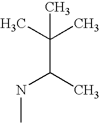 | —C(Cl)═CH—CHO | 5.98 |

TABLE 3

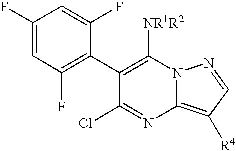

| Ex. No. | 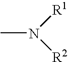 | R⁴ | logP (pH 2.3) |
|---|---|---|---|
| 30 | 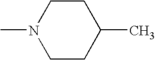 | —CH═CH₂ | |
| 31 | 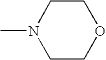 | —CH═CH₂ | |
| 32 | 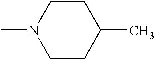 | —CH═CH—COOH | |
| 33 | 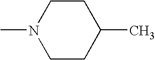 | —CH═CH—COOC₂H₅ | |
| 34 | 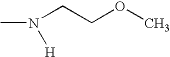 | —CH═CH₂ | |
| 35 | 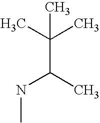 | —CH═CH₂ | |
| 36 | 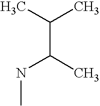 | —CH═CH₂ | |
| 37 | 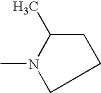 | —CH═CH₂ | |
| 38 | 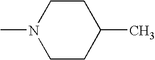 | —C≡CH | |
| 39 | 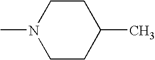 | —CH═CH—CH(CH₃)₂ | |
| 40 | 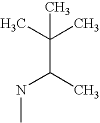 | —C(Cl)═CH—CHO | |

TABLE 4

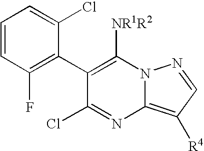

| Ex. No. | 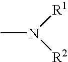 | R⁴ | logP (pH 2.3) |
|---|---|---|---|
| 41 | 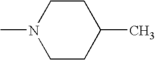 | —CH═CH₂ | |
| 42 | 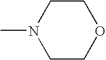 | —CH═CH₂ | |
| 43 | 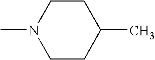 | —CH═CH—COOH | |
| 44 | 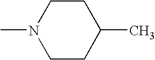 | —CH═CH—COOC₂H₅ | |
| 45 | 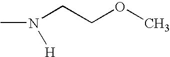 | —CH═CH₂ | |
| 46 | 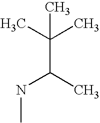 | —CH═CH₂ | |
| 47 | 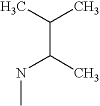 | —CH═CH₂ | |
| 48 | 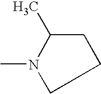 | —CH═CH₂ | |
| 49 | 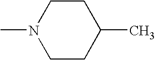 | —C≡CH | |
| 50 | 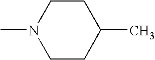 | —CH═CH—CH(CH₃)₂ | |
| 51 | 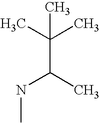 | —C(Cl)═CH—CHO | |

TABLE 5

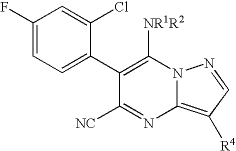

| Ex. No. | 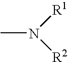 | R⁴ | logP (pH 2.3) |
|---|---|---|---|
| 52 | 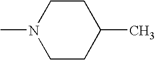 | —CH═CH₂ | |
| 53 | 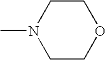 | —CH═CH₂ | |
| 54 | 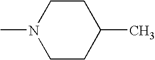 | —CH═CH—COOH | |
| 55 | 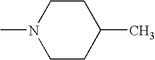 | —CH═CH—COOC₂H₅ | |
| 56 | 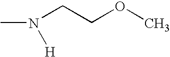 | —CH═CH₂ | |
| 57 | 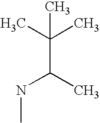 | —CH═CH₂ | |
| 58 | 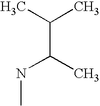 | —CH═CH₂ | |
| 59 | 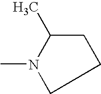 | —CH═CH₂ | |
| 60 | 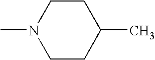 | —C≡CH | |
| 61 | 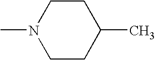 | —CH═CH—CH(CH₃)₂ | |
| 62 | 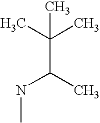 | —C(Cl)═CH—CHO | |

TABLE 6

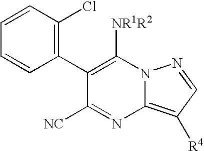

| Ex. No. | 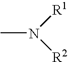 | R⁴ | logP (pH 2.3) |
|---|---|---|---|
| 63 | 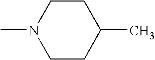 | —CH═CH₂ | |
| 64 | 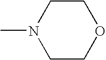 | —CH═CH₂ | |
| 65 | 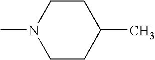 | —CH═CH—COOH | |
| 66 | 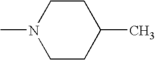 | —CH═CH—COOC₂H₅ | |
| 67 | 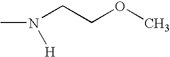 | —CH═CH₂ | |
| 68 | 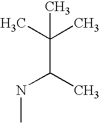 | —CH═CH₂ | |
| 69 | 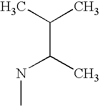 | —CH═CH₂ | |
| 70 | 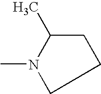 | —CH═CH₂ | |
| 71 | 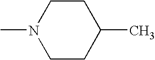 | —C≡CH | |
| 72 | 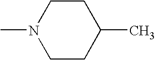 | —CH═CH—CH(CH₃)₂ | |
| 73 | 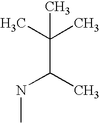 | —C(Cl)═CH—CHO | |

TABLE 7

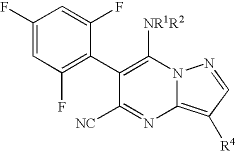

| Ex. No. | 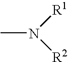 | R⁴ | logP (pH 2.3) |
|---|---|---|---|
| 74 | 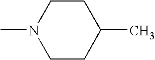 | —CH═CH₂ | |
| 75 | 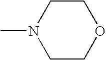 | —CH═CH₂ | |
| 76 | 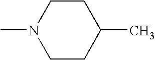 | —CH═CH—COOH | |
| 77 | 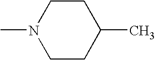 | —CH═CH—COOC₂H₅ | |
| 78 | 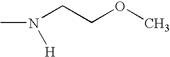 | —CH═CH₂ | |
| 79 | 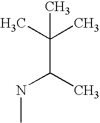 | —CH═CH₂ | |
| 80 | 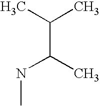 | —CH═CH₂ | |
| 81 | 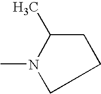 | —CH═CH₂ | |
| 82 | 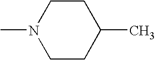 | —C≡CH | |
| 83 | 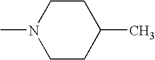 | —CH═CH—CH(CH₃)₂ | |
| 84 | 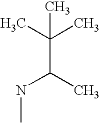 | —C(Cl)═CH—CHO | |

TABLE 8

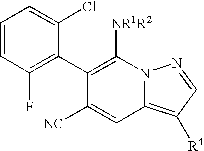

| Ex. No. | 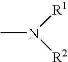 | R⁴ | logP (pH 2.3) |
|---|---|---|---|
| 85 | 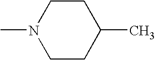 | —CH═CH₂ | |
| 86 | 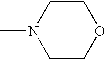 | —CH═CH₂ | |
| 87 | 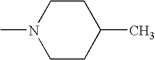 | —CH═CH—COOH | |
| 88 | 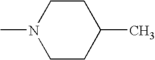 | —CH═CH—COOC₂H₅ | |
| 89 | 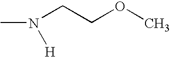 | —CH═CH₂ | |
| 90 | 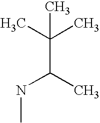 | —CH═CH₂ | |
| 91 | 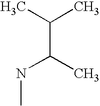 | —CH═CH₂ | |
| 92 | 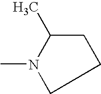 | —CH═CH₂ | |
| 93 | 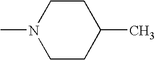 | —C≡CH | |
| 94 | 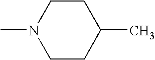 | —CH═CH—CH(CH₃)₂ | |
| 95 | 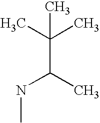 | —C(Cl)═CH—CHO | |

TABLE 9

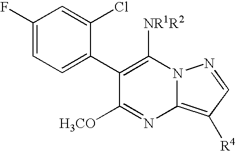

| Ex. No. | 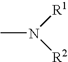 | R⁴ | logP (pH 2.3) |
|---|---|---|---|
| 96 | 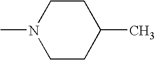 | —CH═CH₂ | |
| 97 | 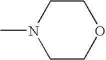 | —CH═CH₂ | |
| 98 | 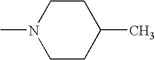 | —CH═CH—COOH | |
| 99 | 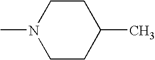 | —CH═CH—COOC₂H₅ | |
| 100 | 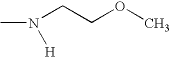 | —CH═CH₂ | |
| 101 | 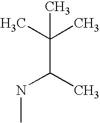 | —CH═CH₂ | |
| 102 | 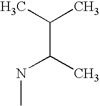 | —CH═CH₂ | |
| 103 | 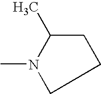 | —CH═CH₂ | |
| 104 | 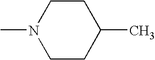 | —C≡CH | |
| 105 | 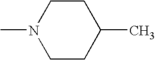 | —CH═CH—CH(CH₃)₂ | |
| 106 | 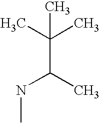 | —C(Cl)═CH—CHO | |

TABLE 10

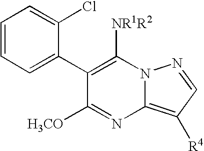

| Ex. No. | 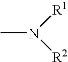 | R⁴ | logP (pH 2.3) |
|---|---|---|---|
| 107 | 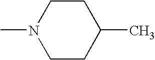 | —CH═CH₂ | |
| 108 | 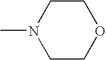 | —CH═CH₂ | |
| 109 | 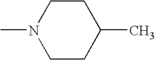 | —CH═CH—COOH | |
| 110 | 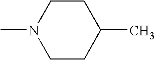 | —CH═CH—COOC₂H₅ | |
| 111 | 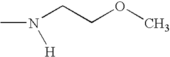 | —CH═CH₂ | |
| 112 | 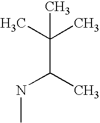 | —CH═CH₂ | |
| 113 | 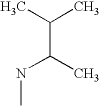 | —CH═CH₂ | |
| 114 | 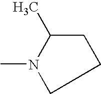 | —CH═CH₂ | |
| 115 | 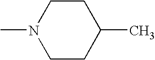 | —C≡CH | |
| 116 | 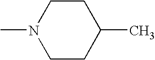 | —CH═CH—CH(CH₃)₂ | |
| 117 | 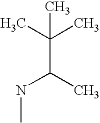 | —C(Cl)═CH—CHO | |

TABLE 11

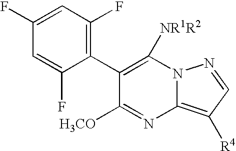

| | | | |
|---|---|---|---|
| Ex. No. | 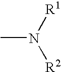 | R⁴ | logP (pH 2.3) |

| | | |
|---|---|---|
| 118 | 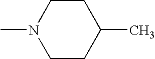 | —CH═CH₂ |
| 119 | 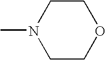 | —CH═CH₂ |
| 120 | 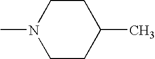 | —CH═CH—COOH |
| 121 | 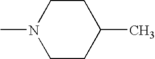 | —CH═CH—COOC₂H₅ |
| 122 | 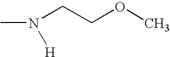 | —CH═CH₂ |
| 123 | 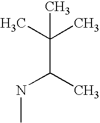 | —CH═CH₂ |
| 124 | 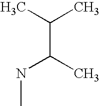 | —CH═CH₂ |
| 125 | 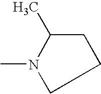 | —CH═CH₂ |
| 126 | 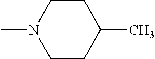 | —C≡CH |
| 127 | 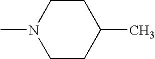 | —CH═CH—CH(CH₃)₂ |
| 128 | 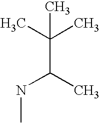 | —C(Cl)═CH—CHO |

TABLE 12

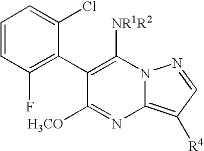

| | | | |
|---|---|---|---|
| Ex. No. | 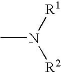 | R⁴ | logP (pH 2.3) |

| | | |
|---|---|---|
| 129 | 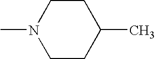 | —CH═CH₂ |
| 130 | 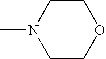 | —CH═CH₂ |
| 131 | 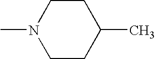 | —CH═CH—COOH |
| 132 | 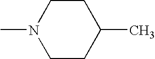 | —CH═CH—COOC₂H₅ |
| 133 | 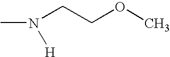 | —CH═CH₂ |
| 134 | 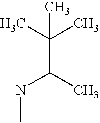 | —CH═CH₂ |
| 135 | 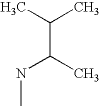 | —CH═CH₂ |
| 136 | 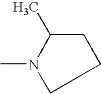 | —CH═CH₂ |
| 137 | 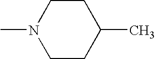 | —C≡CH |
| 138 | 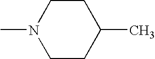 | —CH═CH—CH(CH₃)₂ |
| 139 | 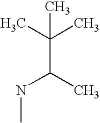 | —C(Cl)═CH—CHO |

The log P values were determined in accordance with EEC Directive 79/831 Annex V. A8 by HPLC (gradient method, acetonitrile/0.1% aqueous phosphoric acid).

Preparation of Starting Materials

### Example 140

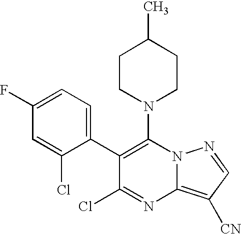

Process (h):

At room temperature, a solution of 5 mmol of 3-cyano-5,7-dichloro-8-(2-chloro-4-fluoro-phenyl)pyrazolo[1,5-a]pyrimidine in 10 ml of acetonitrile is added dropwise with stirring to a mixture of 5 mmol of 4-methylpiperidine and 5 mmol of potassium carbonate in 40 ml of absolute acetonitrile. After the addition has ended, the reaction mixture is stirred at room temperature for another 15 hours and then poured into water. The mixture formed is extracted three times with in each case 30 ml of ethyl acetate. The combined organic phases are dried over sodium sulfate and then concentrated under reduced pressure. This gives 1.73 g of 3-cyano-5-chloro-6-(2-chloro-4-fluorophenyl)-7-(4-methylpiperidino)pyrazolo[1,5-a]pyrimidine.

log P_{(2.3)}=4.88

### Example 141

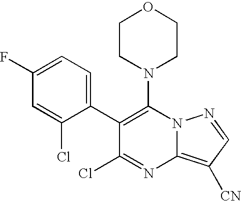

The compound of the formula shown above is prepared according to the method described in example 140.

HPLC: log P=3.33

### Example 142

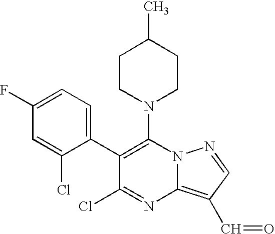

Process (e):

Under an atmosphere of argon, 11 mmol of 3-cyano-5-chloro-6-(2-chloro-4-fluorophenyl)-7-(4-methylpiperidino)pyrazolo[1,5-a]pyrimidine are dissolved in 150 ml of dichloromethane, and 12 mmol of diisobutylaluminum hydride (as a 1 molar solution in toluene) are added with stirring at −50° C. The reaction mixture is stirred at −50° C. for another 30 minutes and then allowed to warm to 0° C., saturated aqueous ammonium chloride solution is added and the mixture is stirred at 0° C. for another 2 hours. 1 N hydrochloric acid is then added, and the organic phase is removed. The aqueous phase is extracted three more times with dichloromethane. The combined organic phases are washed successively with aqueous sodium bicarbonate solution and with aqueous sodium chloride solution, then dried over sodium sulfate and subsequently concentrated under reduced pressure. The residue that remains is chromatographed on silica gel using cyclohexane:ethyl acetate=9:1. This gives 1.73 g of 3-formyl-5-chloro-6-(2-chloro-4-fluorophenyl)-7-(4-methylpiperidino)pyrazolo[1,5-a]pyrimidine.

log P_{(2.3)}−4.53

### Example 143

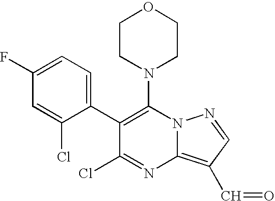

The compound of the formula shown above is prepared according to the method described in example 142.

HPLC: log P=2.94

### Example 144

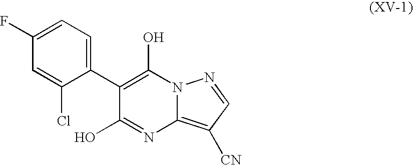

Process (j):

48 g (0.184 mol) of dimethyl 2-chloro-4-fluorophenylmalonate are mixed with 19.91 g (0.184 mol) of 4-cyano-5-aminopyrazole and with 37.55 g (0.203 mol) of tri-n-butylamine, and the mixture is stirred at 180° C. for 6 hours. The methanol formed during the reaction is distilled off. The reaction mixture is then cooled to room temperature. At 95° C. and 1 mbar, volatile components are distilled off. The residue obtained is 6-(2-chloro-4-fluorophenyl)-5,7-dihydroxypyrazolo[1,5-a]pyrimidine-3-carbonitrile in the form of a crude product which is used without additional purification for further syntheses.

### Example 145

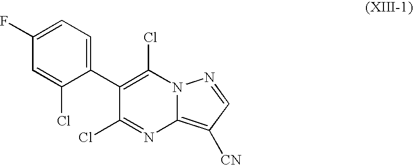

Process (i):

The crude 6-(2-chloro-4-fluorophenyl)-5,7-dihydroxypyrazolo[1,5-a]pyrimidine-3-carbonitrile obtained according to example 10 is dissolved in 367.3 g (2.395 mol) of phosphorus oxychloride. At room temperature, 31.95 g (0.153 mol) of phosphorus pentachloride are added a little at a time. The mixture is then boiled under reflux for 12 hours. The volatile components are distilled off under reduced pressure, dichloromethane is added to the residue and the mixture is washed with water. The organic phase is dried over sodium sulfate and concentrated under reduced pressure. The residue is chromatographed on silica gel using 3 parts of cyclohexane and 1 part of ethyl acetate as mobile phase. This gives 21 g of 95.7% pure 3-cyano-5,7-dichloro-6-(2-chloro-4-fluorophenyl)pyrazolo[1 ,5-a]pyrimidine.

HPLC: log P=3.48

¹H-NMR (DMSO-d6, tetramethylsilane): δ=7.44-7.52 (1H); 7.62-7.66 (1H); 7.71-7.77 (1H); 9.03 (1H) ppm.

### USE EXAMPLES

### Example A

Venturia Test (Apple)/Protective

| | |
|---|---|
| Solvents: | 24.5 parts by weight of acetone |
| | 24.5 parts by weight of dimethylacetamide |
| Emulsifier: | 1.0 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for one day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

In this test, the compounds according to the invention listed in examples 1, 2, 3, 4, 6 and 7 showed, at an application rate of 100 g/ha, an efficacy of more than 80%.

### Example B

Botrytis Test (Bean)/Protective

| | |
|---|---|
| Solvents: | 24.5 parts by weight of acetone |
| | 24.5 parts by weight of dimethylacetamide |
| Emulsifier: | 1.0 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, 2 small pieces of agar colonized by Botrytis cinerea are placed onto each leaf. The inoculated plants are placed in a dark chamber at about 20° C. and 100% relative atmospheric humidity.

2 days after the inoculation, the size of the infected areas on the leaves is evaluated. 0% means an efficacy which corresponds to that of the control, whereas an efficacy of 100% means that no infection is observed.

In this test, the compounds according to the invention listed in examples 1, 2, 3, 4, 6 and 7 showed, at an application rate of 100 g/ha, an efficacy of at least 90%.

### Example C

Alternaria Test (Tomato)/Potective

| | |
|---|---|
| Solvent: | 49 parts by weight of N,N-dimethylformamide |
| Emulsifier: | 1 part by weight of alkylaryl glycol ether |

To produce a suitable preparation of active compound, 1 part by weight of active compound is mixed with the stated amounts of solvent and emulsifier, and the concentrate is diluted with water to the desired concentration.

To test for protective activity, young tomato plants are sprayed with the preparation of active compound at the stated application rate. 1 day after the treatment, the plants are inoculated with a spore suspension of Alternaria solani and then remain at 100% relative atmospheric humidity and 20° C. for 24 h. The plants then remain at 96% relative atmospheric humidity and a temperature of 20° C.

Evaluation is carried out 7 days after the inoculation. 0% means an efficacy which corresponds to that of the control, whereas an efficacy of 100% means that no infection is observed.

In this test, the compounds according to the invention listed in examples 1, 6 and 7 showed, at an application rate of 750 g/ha, an efficacy of at least 90%.

### Example D

Podosphaera Test (Apple)/Protective

| | |
|---|---|
| Solvents: | 24.5 parts by weight of acetone |
| | 24.5 parts by weight of dimethylacetamide |
| Emulsifier: | 1.0 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of the apple mildew pathogen Podosphaera leucotricha. The plants are then placed in a greenhouse at 23° C. and a relative atmospheric humidity of about 70%.

Evaluation is carried out 10 days after the inoculation. 0% means an efficacy which corresponds to that of the control, whereas an efficacy of 100% means that no infection is observed.

In this test, the compounds according to the invention listed in examples 1, 2, 3, 4, 6 and 7 showed, at an application rate of 1000 g/ha, an efficacy of at least 80%.

1. A pyrazolopyrimidine of the formula 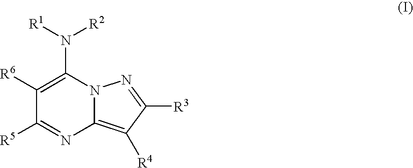
in which
R¹ represents optionally substituted alkyl, optionally substituted alkenyl, optionally substituted alkynyl, optionally substituted cycloalkyl or optionally substituted heterocyclyl,
R² represents hydrogen or alkyl, or
R¹ and R² together with the nitrogen atom to which they are attached represent an optionally substituted heterocyclic ring,
R³ represents hydrogen or alkyl,
R⁴ represents optionally substituted alkenyl or optionally substituted alkynyl,
R⁵ represents halogen, cyano, alkyl, alkoxy or alkylthio and
R⁶ represents alkyl, cycloalkyl or optionally substituted aryl.

2. A pyrazolopyrimidine according to claim 1 in which
R¹ represents alkyl having 1 to 6 carbon atoms which is optionally mono- to pentasubstituted by identical or different substituents selected from the group consisting of halogen, cyano, hydroxyl, alkoxy having 1 to 4 carbon atoms and cycloalkyl having 3 to 6 carbon atoms, or
R¹ represents alkenyl having 2 to 6 carbon atoms which is optionally mono- to trisubstituted by identical or different substituents selected from the group consisting of halogen, cyano, hydroxyl, alkoxy having 1 to 4 carbon atoms and cycloalkyl having 3 to 6 carbon atoms, or
R¹ represents alkynyl having 3 to 6 carbon atoms which is optionally mono- to trisubstituted by identical or different substituents selected from the group consisting of halogen, cyano, alkoxy having 1 to 4 carbon atoms and cycloalkyl having 3 to 6 carbon atoms, or
R¹ represents cycloalkyl having 3 to 6 carbon atoms which is optionally mono- to trisubstituted by identical or different substituents selected from the group consisting of halogen and alkyl having 1 to 4 carbon atoms, or
R¹ represents saturated or unsaturated heterocycle having 5 or 6 ring members and 1 to 3 heteroatoms, where the heterocycle is optionally mono- or disubstituted by halogen, alkyl having 1 to 4 carbon atoms, cyano, nitro or cycloalkyl having 3 to 6 carbon atoms,
R² represents hydrogen or alkyl having 1 to 4 carbon atoms, or
R¹ and R² together with the nitrogen atom to which they are attached represent a saturated or unsaturated heterocyclic ring having 3 to 6 ring members, where the heterocycle may contain a further nitrogen, oxygen or sulfur atom as ring member and where the heterocycle is optionally mono- to tri-substituted by fluorine, chlorine, bromine, alkyl having 1 to 4 carbon atoms or haloalkyl having 1 to 4 carbon atoms and 1 to 9 fluorine or chlorine atoms,
R³ represents hydrogen or alkyl having 1 to 4 carbon atoms,
R⁴ represents alkenyl having 2 to 6 carbon atoms or alkynyl having 2 to 6 carbon atoms, or
R⁴ represents alkenyl having 2 to 4 carbon atoms which is substituted by carboxyl, methoxycarbonyl, ethoxycarbonyl, formyl or halogen, or represents alkynyl having 2 to 4 carbon atoms which is substituted by carboxyl, methoxycarbonyl or ethoxycarbonyl, formyl or halogen,
R⁵ represents fluorine, chlorine, bromine, cyano, alkoxy having 1 to 4 carbon atoms or alkylthio having 1 to 4 carbon atoms and
R⁶ represents alkyl having 1 to 6 carbon atoms or represents cycloalkyl having 3 to 6 carbon atoms, or represents phenyl which is optionally mono- to tetrasubstituted by identical or different substituents.

3. A pyrazolopyrimidine according to claim 1 in which
R¹ represents a radical of the formula 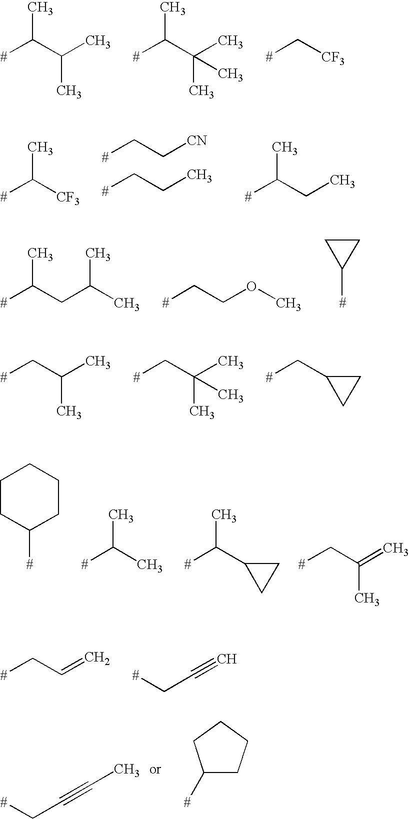
where # denotes the point of attachment, and stereoisomers thereof,
R² represents hydrogen, methyl, ethyl or propyl, or
R¹ and R² together with the nitrogen atom to which they are attached represent pyrrolidinyl, piperidinyl, morpholinyl, thiomorpholinyl, piperazinyl, 3,6-dihydro-1(2H)-piperidinyl or tetrahydro-1(2H)-pyridazinyl, where each of these radicals is optionally substituted by 1 to 3 fluorine atoms, 1 to 3 methyl groups or trifluoromethyl, or
R¹ and R² together with the nitrogen atom to which they are attached represent a radical of the formula 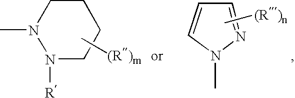
in which
R′ represents hydrogen or methyl,
R″ represents methyl, ethyl, fluorine, chlorine or trifluoromethyl,
m represents the number 0, 1, 2 or 3, where R″ represents identical or different radicals if m represents 2 or 3,
R′″ represents methyl, ethyl, fluorine, chlorine or trifluoromethyl and
n represents the number 0, 1, 2 or 3, where R′″ represents identical or different radicals if n represents 2 or 3,
R³ represents hydrogen, methyl, ethyl, propyl or isopropyl,
R⁴ represents straight-chain or branched alkenyl having 2 to 5 carbon atoms, where each of these radicals is optionally monosubstituted by carboxyl, methoxycarbonyl, ethoxycarbonyl, formyl or halogen, or
R⁴ represents alkynyl having 2 to 5 carbon atoms, where each of these radicals is optionally monosubstituted by carboxyl, methoxycarbonyl or ethoxycarbonyl,
R⁵ represents fluorine, chlorine, cyano, methoxy, ethoxy, methylthio or ethylthio, and
R⁶ represents straight-chain or branched alkyl having 1 to 4 carbon atoms, cyclopropyl, cyclobutyl, cyclopentyl or cyclohexyl, or
R⁶ represents phenyl which is optionally mono- to trisubstituted by identical or different substituents selected from the group consisting of fluorine, chlorine, bromine, cyano, nitro, formyl, methyl, ethyl, propyl, butyl, allyl, propargyl, methoxy, ethoxy, propoxy, methylthio, ethylthio, propylthio, methylsulfinyl, ethylsulfinyl, methylsulfonyl, ethylsulfonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulfinyl, trifluoromethylsulfonyl, trichloroethynyloxy, trifluoroethynyloxy, chloroallyloxy, iodopropargyloxy, methylamino, ethylamino, propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl, ethoximinoethyl, cyclopropyl, cyclobutyl, cyclopentyl and cyclohexyl, 2,3-attached 1,3-propanediyl, methylenedioxy (—O—CH₂—O—) or 1,2-ethylenedioxy (—O—CH₂—CH₂—O—), where each of these radicals is optionally mono- or polysubstituted by identical or different substituents selected from the group consisting of fluorine, chlorine, methyl, ethyl, propyl and trifluoromethyl.

4. A pyrazolopyrimidine according to claim 1 in which
R⁴ represents a radical of the formula 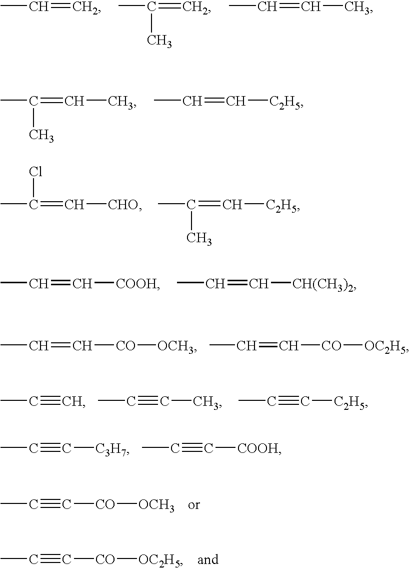
R⁶ represents methyl, ethyl, propyl, isopropyl, n-butyl, tert-butyl, cyclopropyl, cyclopentyl or cyclohexyl, or
R⁶ represents 2,4-, 2,5- or 2,6-disubstituted phenyl, 2-substituted phenyl or 2,4,6-trisubstituted phenyl, where the substituents are selected from the group consisting of fluorine, chlorine, bromine, cyano, nitro, formyl, methyl, ethyl, propyl, butly, allyl, propargyl, methoxy, ethoxy, propoxy, methylthio, ethylthio, propylthio, methylsulfinyl, ethylsulfinyl, methylsulfonyl, ethylsulfonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulfinyl, trifluoromethylsulfonyl, trichloroethynyloxy, trifluoroethynyloxy, chloroallyloxy, iodopropargyloxy, methylamino, ethylamino, propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl, ethoximinoethyl, cyclopropyl, cyclobutyl, cyclopentyl and cyclohexyl, 2,3-attached 1,3-propanediyl, methylenedioxy (—O—CH₂—O—) or 1,2-ethylenedioxy (—O—CH₂—CH₂—O—), where each of these radicals is optionally mono- or polysubstituted by identical or different substituents selected from the group consisting of fluorine, chlorine, methyl, ethyl, propyl and trifluoromethyl.

5. A process for preparing pyrazolopyrimidines of the formula (I) according to claim 1, comprising
(a) reacting a pyrazolopyrimidine of the formula 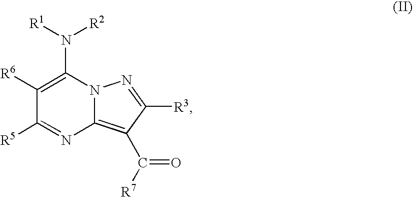
in which
R¹, R², R³, R⁵ and R⁶ are as defined in claim 1, and
R⁷ represents hydrogen or alkyl with a phosphonium salt of the formula
Y₃^{⊕}P—CH₂—R⁸ X^{⊖} (III)
in which
Y represents alkyl, cycloalkyl, aralkyl or phenyl,
X represents an anion, such as bromide, and
R⁸ represents hydrogen or optionally substituted alkyl in the presence of a base and a diluent, or
(b) reacting a pyrazolopyrimidine of the formula 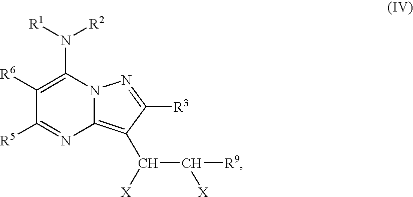
in which
R¹, R², R³, R⁵ and R⁶ are as defined in claim 1,
R⁹ represents hydrogen or optionally substituted alkyl,
X represents chlorine or bromine with a strong base in the presence of a diluent, or
(c) reacting a pyrazolopyrimidine of the formula 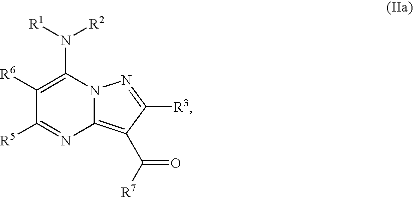
in which
R¹, R², R³, R⁵, R⁶ are as defined in claim 1, and
R⁷ represents hydrogen or alkyl with phosphorus oxychloride in the presence of dimethylformamide and a base to give a compound of the formula (V) 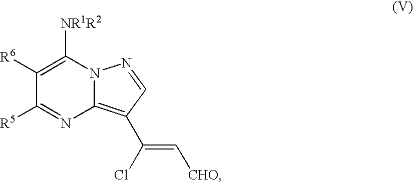
or
(d) reacting a pyrazolopydrimidine of the formula 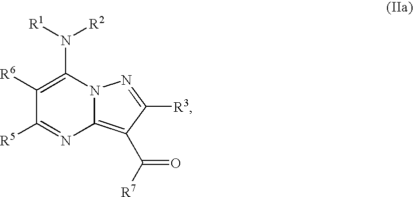
in which
R¹, R², R³, R⁵, R⁶ are as defined in claim 1, and
R⁷ represents hydrogen or alkyl with a Grignard compound
R⁸—CH₂—Mg X,
where R⁸ represents hydrogen or optionally substituted alkyl, and then an acid.

6. A composition for controlling unwanted microorganisms, comprising a mixture of at least one pyrazolopyrimidine according to claim 1, and one or more extenders or surfactants.

7. The composition according to claim 6, further comprising at least one fungicidally or insecticidally active compound.

8. (canceled)

9. A method for controlling unwanted microorganisms, comprising contacting unwanted microorganisms or their habitat with a pyrazolopyrimidine of the formula (I) according to claim 1.

10. A process for preparing compositions for controlling unwanted microorganisms, comprising mixing a pyrazolopyrimidine of the formula (I) according to claim 1 with an extender, surfactant or combination thereof.

11. A pyrazolopyrimidine according to claim 1 in which
R¹ represents a radical of the formula 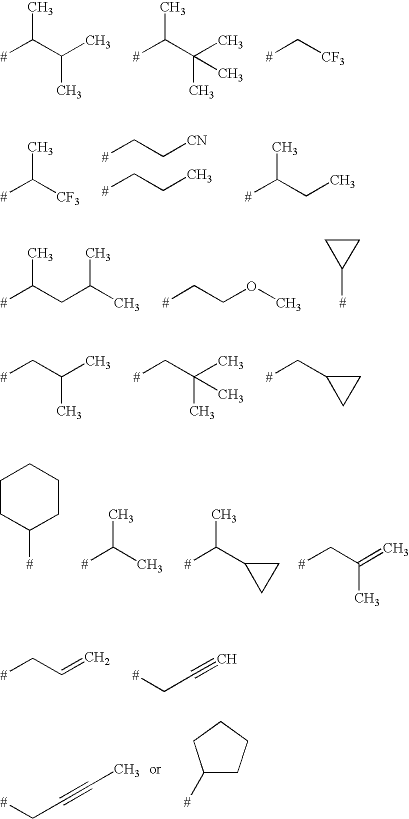
where # denotes the point of attachment and stereoisomers thereof,
R² represents hydrogen, methyl, ethyl or propyl, or
R¹ and R² together with the nitrogen atom to which they are attached represent pyrrolidinyl, piperidinyl, morpholinyl, thiomorpholinyl, piperazinyl, 3,6-dihydro-1(2H)-piperidinyl or tetrahydro-1(2H)-pyridazinyl, where each of these radicals is optionally substituted by 1 to 3 fluorine atoms, 1 to 3 methyl groups or trifluoromethyl, or
R¹ and R² together with the nitrogen atom to which they are attached represent a radical of the formula 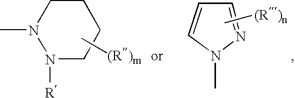
in which
R′ represents hydrogen or methyl,
R″ represents methyl, ethyl, fluorine, chlorine or trifluoromethyl,
m represents the number 0, 1, 2 or 3, where R″ represents identical or different radicals if m represents 2 or 3,
R′″ represents methyl, ethyl, fluorine, chlorine or trifluoromethyl and
n represents the number 0, 1, 2 or 3, where R′″ represents identical or different radicals if n represents 2 or 3,
R³ represents hydrogen, methyl, ethyl, propyl or isopropyl,
R⁴ represents a radical of the formula 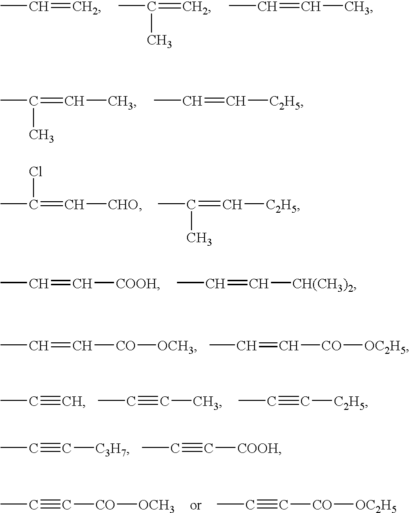
and
R⁶ represents methyl, ethyl, propyl, isopropyl, n-butyl, tert-butyl, cyclopropyl, cyclopentyl or cyclohexyl, or
R⁶ represents 2,4-, 2,5- or 2,6-disubstituted phenyl, 2-substituted phenyl or 2,4,6-trisubstituted phenyl, where the substituents are selected from the group consisting of fluorine, chlorine, bromine, cyano, nitro, formyl, methyl, ethyl, propyl, butyl, allyl, propargyl, methoxy, ethoxy, propoxy, methylthio, ethylthio, propylthio, methylsulfinyl, ethylsulfinyl, methylsulfonyl, ethylsulfonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulfinyl, trifluoromethylsulfonyl, trichloroethynyloxy, trifluoroethynyloxy, chloroallyloxy, iodopropargyloxy, methylamino, ethylamino, propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl, ethoximinoethyl, cyclopropyl, cyclobutyl, cyclopentyl and cyclohexyl, 2,3-attached 1,3-propanediyl, methylenedioxy (—O—CH₂—O—) or 1,2-ethylenedioxy (—O—CH₂—CH₂—O—), where each of these radicals is optionally mono- or polysubstituted by identical or different substituents selected from the group consisting of fluorine, chlorine, methyl, ethyl, propyl and trifluoromethyl.
